(19) United States
(12) Patent Application Publication
(10) Pub. No.: US 2011/0039824 A1
(43) Pub. Date: Feb. 17, 2011

(54) 1,2,3,6-tetrahydroazepino[4,5-b]indole-5-carboxylate nuclear receptor inhibitors

(75) Inventors: Joseph Theodore Lundquist, IV, Limerick, PA (US); Paige Erin Mahaney, Pottstown, PA (US); Callain Younghee Kim, Collegeville, PA (US); Matthew Lantz Crawley, Wayne, PA (US)

Correspondence Address: WYETH LLC/FINNEGAN HENDERSON, LLP, 901 NEW YORK AVENUE, NW, WASHINGTON, DC, 20001-4413, US

(73) Assignee: Wyeth, Madison, NJ (US)

(21) Appl. No.: 12/662,971

(22) Filed: May 13, 2010

Related U.S. Application Data
(63) Continuation of application No. 12/567,282, filed on Sep. 25, 2009, now abandoned.
(60) Provisional application No. 61/100,644, filed on Sep. 26, 2008.

(51) Int. Cl. A61K 31/55; C07D 487/04; A61P 3/06; A61P 3/04; A61P 3/10; A61P 1/16; A61P 35/00; A61P 9/10; C12N 5/02

(52) U.S. Cl. 514/215; 540/580; 435/375

(57) ABSTRACT

Provided are certain 1,2,3,6-tetrahydroazepino[4,5-b]indole-5-carboxylate compounds which are useful for modulating the activity of nuclear receptors, such as farnesoid X receptors, and/or for the treatment, prevention, or amelioration diseases or disorders related to the activity of these receptors.

This application claims the benefit of priority to U.S. Provisional Application No. 61/100,644, filed Sep. 26, 2008, the disclosure of which is incorporated herein by reference.

Provided are certain 1,2,3,6-tetrahydroazepino[4,5-b]indole-5-carboxylate compounds which may be useful for modulating the activity of nuclear receptors, such as farnesoid X receptors, and/or for the treatment, prevention, or amelioration diseases or disorders related to the activity of these receptors.

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin and mineralocorticoid receptors which bind as homodimers to hormone response elements (HREs) organized as inverted repeats (see, e.g., Glass, supra). A second class of receptors, including those activated by retinoic acid, thyroid hormone, vitamin D₃, fatty acids/peroxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al. (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al. (1992) Genes Dev. 6:329-344), coding for RXRα, -β, and -γ, all of which are able to heterodimerize with any of the class II receptors, although there appear to be preferences for distinct RXR subtypes by partner receptors in vivo (see, e.g., Chiba et al. (1997) Mol. Cell. Biol. 17:3013-3020). In the adult liver, RXRα is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al. (2000) Mol. Cell. Biol 20:4436-4444.

Included in the nuclear receptor superfamily of regulatory proteins are nuclear receptors for which the ligand is known and those which lack known ligands. Nuclear receptors falling in the latter category are referred to as orphan nuclear receptors. The search for activators for orphan receptors has led to the discovery of previously unknown signaling pathways (see, e.g., Levin et al., (1992), supra and Heyman et al., (1992), supra). For example, it has been reported that bile acids, which are involved in physiological processes such as cholesterol catabolism, are ligands for the farnesoid X receptor (infra).

Since it is known that products of intermediary metabolism act as transcriptional regulators in bacteria and yeast, such molecules may serve similar functions in higher organisms (see, e.g., Tomkins (1975) Science 189:760-763 and O'Malley (1989) Endocrinology 125:1119-1120). For example, one biosynthetic pathway in higher eukaryotes is the mevalonate pathway, which leads to the synthesis of cholesterol, bile acids, porphyrin, dolichol, ubiquinone, carotenoids, retinoids, vitamin D, steroid hormones and farnesylated proteins.

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is primarily expressed in the liver, kidney and intestine (see, e.g., Seol et al., supra and Forman et al. (1995) Cell 81:687-693). It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317.

Nuclear receptor activity, including the farnesoid X receptor and/or orphan nuclear receptor activity, has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis and xanthoma, (see, e.g., International Patent Application Publication No. WO 00/57915), hyperlipoproteinemia (see, e.g., International Patent Application Publication No. WO 01/60818), hypertriglyceridemia, lipodystrophy, peripheral occlusive disease, ischemic stroke, hyperglycemia and diabetes mellitus (see, e.g., International Patent Application Publication No. WO 01/82917), disorders related to insulin resistance including the cluster of disease states, conditions or disorders that make up “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), obesity, acne (see, e.g., International Patent Application Publication No. WO 00/49992), and cancer, cholestasis, Parkinson's disease and Alzheimer's disease (see, e.g., International Patent Application Publication No. WO 00/17334).

The activity of nuclear receptors, including the farnesoid X receptor and/or orphan nuclear receptors, has been implicated in physiological processes including, but not limited to, triglyceride metabolism, catabolism, transport or absorption, bile acid metabolism, catabolism, transport, absorption, re-absorption or bile pool composition, cholesterol metabolism, catabolism, transport, absorption, or re-absorption. The modulation of cholesterol 7α-hydroxylase gene (CYP7A1) transcription (see, e.g., Chiang et al. (2000) J. Biol. Chem. 275:10918-10924), HDL metabolism (see, e.g., Urizar et al. (2000) J. Biol. Chem. 275:39313-39317), hyperlipidemia, cholestasis, and increased cholesterol efflux and increased expression of ATP binding cassette transporter protein (ABC1) (see, e.g., International Patent Application Publication No. WO 00/78972) are also modulated or otherwise affected by the farnesoid X receptor.

Thus, there is a need for compounds, pharmaceutical compositions and methods of modulating the activity of nuclear receptors, including the farnesoid X receptor and/or orphan nuclear receptors. Such compounds and pharmaceutical compositions may be useful in the treatment, prevention, or amelioration of one or more symptoms of diseases or disorders in which nuclear receptor activity is implicated.

Provided is a compound of Formula I

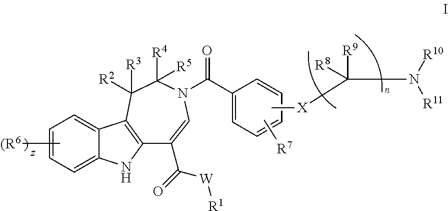

or a pharmaceutically acceptable salt thereof, wherein
W is chosen from O and NH;
X is chosen from O and CR⁸R⁹;
n is 2, 3 or 4 when X is equal to O, or
n is 0, 1, 2, 3 or 4 when X is equal to CR⁸R⁹;
z is 1 or 2;
R¹ is chosen from optionally substituted alkyl, optionally substituted cycloalkyl, optionally substituted heterocyclyl, optionally substituted aryl, and optionally substituted heteroaryl;
R², R³, R⁴, and R⁵ are independently chosen from hydrogen and optionally substituted alkyl, or any two of R², R³, R⁴ and R⁵, together with the atoms to which they are attached, form an optionally substituted cycloalkyl or optionally substituted heterocyclyl ring;
R⁶, at each occurrence, independently is chosen from halogen, optionally substituted alkyl, hydroxyl, optionally substituted alkoxy and cyano;
R⁷ is chosen from hydrogen, halogen, optionally substituted alkyl, hydroxyl, optionally substituted alkoxy and cyano;
R⁸ and R⁹, at each occurrence, are independently chosen from hydrogen, fluoro, and alkyl; and
R¹⁰ and R¹¹ are independently chosen from hydrogen, optionally substituted alkyl, optionally substituted cycloalkyl and optionally substituted heterocyclyl, or R¹⁰ and R¹¹ together with the atoms to which they are attached, form an optionally substituted heterocyclyl ring containing 1 or 2 heteroatoms including the nitrogen through which they are attached.

Also provided is a compound chosen from
isopropyl 1,1-dimethyl-3-[4-(3-pyrrolidin-1-ylpropoxy)benzoyl]-1,2,3,6-tetrahydroazepino[4,5-b]indole-5-carboxylate;
isopropyl 1,1-dimethyl-3-{4-[2-(4-methylpiperazin-1-yl)ethoxy]benzoyl}-1,2,3,6-tetrahydroazepino[4,5-b]indole-5-carboxylate;
isopropyl 1,1-dimethyl-3-{4-[3-(4-methylpiperazin-1-yl)propoxy]benzoyl}-1,2,3,6-tetrahydroazepino[4,5-b]indole-5-carboxylate;
isopropyl 1,1-dimethyl-3-[3-(2-pyrrolidin-1-ylethoxy)benzoyl]-1,2,3,6-tetrahydroazepino[4,5-b]indole-5-carboxylate;
isopropyl 1,1-dimethyl-3-{3-[2-(4-methylpiperazin-1-yl)ethoxy]benzoyl}-1,2,3,6-tetrahydroazepino[4,5-b]indole-5-carboxylate;
isopropyl 1,1-dimethyl-3-[3-(3-pyrrolidin-1-ylpropoxy)benzoyl]-1,2,3,6-tetrahydroazepino[4,5-b]indole-5-carboxylate;
isopropyl 1,1-dimethyl-3-{3-[3-(4-methylpiperazin-1-yl)propoxy]benzoyl}-1,2,3,6-tetrahydroazepino[4,5-b]indole-5-carboxylate;
isopropyl 1,1-dimethyl-3-[4-(morpholin-4-ylmethyl)benzoyl]-1,2,3,6-tetrahydroazepino[4,5-b]indole-5-carboxylate;
isopropyl 1,1-dimethyl-3-[4-(2-morpholin-4-ylethyl)benzoyl]-1,2,3,6-tetrahydroazepino[4,5-b]indole-5-carboxylate;
isopropyl 3-(3-{[(2R,6R)-2,6-dimethylmorpholin-4-yl]methyl}benzoyl)-1,1-dimethyl-1,2,3,6-tetrahydroazepino[4,5-b]indole-5-carboxylate;
isopropyl 3-(3-{[(2R,6S)-2,6-dimethylmorpholin-4-yl]methyl}benzoyl)-1,1-dimethyl-1,2,3,6-tetrahydroazepino[4,5-b]indole-5-carboxylate;
isopropyl 3-(3-{[(3R,5S)-3,5-dimethylpiperazin-1-yl]methyl}benzoyl)-1,1-dimethyl-1,2,3,6-tetrahydroazepino[4,5-b]indole-5-carboxylate;
isopropyl 3-{3-[(4-carbamoylpiperidin-1-yl)methyl]benzoyl}-1,1-dimethyl-1,2,3,6-tetrahydroazepino[4,5-b]indole-5-carboxylate;
isopropyl 1,1-dimethyl-3-{3-[(4-morpholin-4-ylpiperidin-1-yl)methyl]benzoyl}-1,2,3,6-tetrahydroazepino[4,5-b]indole-5-carboxylate;
isopropyl 1,1-dimethyl-3-[3-(1,3-thiazolidin-3-ylmethyl)benzoyl]-1,2,3,6-tetrahydroazepino[4,5-b]indole-5-carboxylate;
isopropyl 3-[3-(1,4′-bipiperidin-1′-ylmethyl)benzoyl]-1,1-dimethyl-1,2,3,6-tetrahydroazepino[4,5-b]indole-5-carboxylate;
isopropyl 1,1-dimethyl-3-{3-[(4-pyrrolidin-1-ylpiperidin-1-yl)methyl]benzoyl}-1,2,3,6-tetrahydroazepino[4,5-b]indole-5-carboxylate;
isopropyl 3-(3-{[(3R,5S)-3,5-dimethylpiperidin-1-yl]methyl}benzoyl)-1,1-dimethyl-1,2,3,6-tetrahydroazepino[4,5-b]indole-5-carboxylate;
isopropyl 3-(3-{[(3S,5S)-3,5-dimethylpiperidin-1-yl]methyl}benzoyl)-1,1-dimethyl-1,2,3,6-tetrahydroazepino[4,5-b]indole-5-carboxylate;
isopropyl 1,1-dimethyl-3-[3-(thiomorpholin-4-ylmethyl)benzoyl]-1,2,3,6-tetrahydroazepino[4,5-b]indole-5-carboxylate;
isopropyl 3-(3-{[(2S,5S)-2,5-dimethylpyrrolidin-1-yl]methyl}benzoyl)-1,1-dimethyl-1,2,3,6-tetrahydroazepino[4,5-b]indole-5-carboxylate;
isopropyl 3-{3-[(cyclohexylamino)methyl]benzoyl}-1,1-dimethyl-1,2,3,6-tetrahydroazepino[4,5-b]indole-5-carboxylate;
isopropyl 3-(3-{[cyclohexyl(methyl)amino]methyl}benzoyl)-1,1-dimethyl-1,2,3,6-tetrahydroazepino[4,5-b]indole-5-carboxylate;
isopropyl 3-{3-[(4-hydroxypiperidin-1-yl)methyl]benzoyl}-1,1-dimethyl-1,2,3,6-tetrahydroazepino[4,5-b]indole-5-carboxylate;
isopropyl 3-{3-[(3-carbamoylpiperidin-1-yl)methyl]benzoyl}-1,1-dimethyl-1,2,3,6-tetrahydroazepino[4,5-b]indole-5-carboxylate;
isopropyl 1,1-dimethyl-3-{3-[(piperidin-4-ylamino)methyl]benzoyl}-1,2,3,6-tetrahydroazepino[4,5-b]indole-5-carboxylate; and
isopropyl 3-{3-[(1,1-dioxidothiomorpholin-4-yl)methyl]benzoyl}-1,1-dimethyl-1,2,3,6-tetrahydroazepino[4,5-b]indole-5-carboxylate,
   or a pharmaceutically acceptable salt thereof.

Also provided is a pharmaceutical composition comprising a therapeutically effective amount of at least one compound or pharmaceutically acceptable salt thereof described herein.

Also provided is a method of treating, preventing, inhibiting, or ameliorating one or more symptoms of a disease or disorder in which nuclear receptor activity is implicated, comprising administering to a subject in need thereof an effective amount of at least one compound or pharmaceutically acceptable salt thereof described herein or at least one pharmaceutical composition described herein.

Also provided is a method of reducing plasma cholesterol levels in a subject in need thereof, comprising administering an effective amount of at least one compound or pharmaceutically acceptable salt thereof described herein or at least one pharmaceutical composition described herein.

Also provided is a method of reducing plasma triglyceride levels in a subject in need thereof, comprising administering an effective amount of at least one compound or pharmaceutically acceptable salt thereof described herein or at least one pharmaceutical composition described herein.

Also provided is a method of treating, preventing, inhibiting or ameliorating one or more symptoms of a disease or disorder which is affected by abnormal cholesterol, triglyceride, or bile acid levels, comprising administering to a subject in need thereof an effective amount of at least one compound or pharmaceutically acceptable salt thereof described herein or at least one pharmaceutical composition described herein.

Also provided is a method of modulating cholesterol metabolism, catabolism, synthesis, absorption, reabsorption, secretion or excretion in a mammal, comprising administering an effective amount of at least one compound or pharmaceutically acceptable salt thereof described herein or at least one pharmaceutical composition described herein.

Also provided is a method of treating at least one malignancy in a patient, the method comprising administering to the patient a therapeutically effective amount of at least one compound or pharmaceutically acceptable salt thereof described herein or at least one pharmaceutical composition described herein, wherein the at least one compound or pharmaceutically acceptable salt thereof or composition induces expression of the reversion-inducing-cysteine rich-protein with Kazal motifs (RECK) gene in the patient.

Also provided is a method of treating nonalcoholic fatty liver disease (NAFLD) in a patient, the method comprising administering to the patient a therapeutically effective amount of at least one compound or pharmaceutically acceptable salt thereof described herein or at least one pharmaceutical composition described herein.

Also provided is a method of treating a patient with existing cholesterol gallstone disease, wherein the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress in the liver, and an elevated level of at least one of VCAM-1, ICAM-1, TNFα, MCP-1, KC, TIMP-1, MMP-9, MMP-14, CYP2E1, ALT, AST, and CK-18, the method comprising administering to the patient a therapeutically effective amount of at least one compound or pharmaceutically acceptable salt thereof described herein or at least one pharmaceutical composition described herein.

Also provided is a method of treating at least one disease state characterized by elevated expression of the Lectin-like Oxidized Low-density Lipoprotein Receptor 1 (LOX-1) in a patient, the method comprising administering to the patient a therapeutically effective amount of at least one compound or pharmaceutically acceptable salt thereof described herein or at least one pharmaceutical composition described herein, wherein the at least one compound or pharmaceutically acceptable salt thereof or composition reduces expression of LOX-1 in the patient.

Also provided is a method of treating at least one condition that can be treated by elevating the vitamin D receptor (VDR) activity level in a patient, the method comprising administering to the patient a therapeutically effective amount of at least one compound or pharmaceutically acceptable salt thereof described herein or at least one pharmaceutical composition described herein, wherein the at least one compound or pharmaceutically acceptable salt thereof or composition elevates the level of Cytochrome P450, family 27, subfamily B, polypeptide 1 (CYP27B1), to thereby elevate the level of VDR activity in the patient.

Also provided is a method for modulating farnesoid X receptor activity comprising contacting a cell with at least one compound or pharmaceutically acceptable salt thereof described herein or at least one pharmaceutical composition described herein.

Unless defined otherwise, technical and scientific terms used herein have the same meaning as is commonly understood by one of ordinary skill in the art to which this invention belongs. In the event that there are a plurality of definitions for a term herein, those in this section prevail unless stated otherwise.

As used herein, a nuclear receptor is a member of a superfamily of regulatory proteins that are receptors for, e.g., steroids, retinoids, vitamin D and thyroid hormones. These proteins bind to cis-acting elements in the promoters of their target genes and modulate gene expression in response to a ligand therefor. Nuclear receptors may be classified based on their DNA binding properties. For example, the glucocorticoid, estrogen, androgen, progestin and mineralocorticoid receptors bind as homodimers to hormone response elements (HREs) organized as inverted repeats. Another example are receptors, including those activated by retinoic acid, thyroid hormone, vitamin D₃, fatty acids/peroxisome proliferators and ecdysone, that bind to HREs as heterodimers with a common partner, the retinoid X receptor (RXR). Among the latter receptors is the farnesoid X receptor.

As used herein, an orphan nuclear receptor is a gene product that embodies the structural features of a nuclear receptor that was identified without any prior knowledge of their association with a putative ligand and/or for which the natural ligand is unknown. Under this definition, orphan nuclear receptors include, without limitation, farnesoid X receptors, liver X receptors (LXR α & β), retinoid X receptors (RXR α, β & γ), and peroxisome proliferator activator receptors (PPAR α, β & γ) (see, Giguere, Endocrine Reviews (1999), Vol. 20, No. 5: pp. 689-725).

As used herein, farnesoid X receptor refers to all mammalian forms of such receptor including, for example, alternative splice isoforms and naturally occurring isoforms (see, e.g. Huber et al, Gene (2002), Vol. 290, pp.: 35-43). Representative farnesoid X receptor species include, without limitation the rat (GenBank Accession No. NM_{—}021745), mouse (Genbank Accession No. NM_{—}009108), and human (GenBank Accession No. NM_{—}005123) forms of the receptor.

As used herein, treatment means any manner in which one or more of the symptoms of a disease or disorder are ameliorated or otherwise beneficially altered. Treatment also encompasses any pharmaceutical use of the compositions herein, such as use for treating a nuclear receptor mediated diseases or disorders, or diseases or disorders in which nuclear receptor activity, including the farnesoid X receptor or orphan nuclear receptor activity, is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of nuclear receptor, including the farnesoid X receptor, activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

The term “therapeutically effective amount” means an amount effective, when administered to a patient, to provide a therapeutic benefit such as amelioration of symptoms, slowing of disease progression, or prevention of disease or injury.

It is to be understood that the compounds provided herein may contain chiral centers. Such chiral centers may be of either the (R) or (S) configuration, or may be a mixture thereof. Thus, the compounds described herein may be enantiomerically pure, or be stereoisomeric or diastereomeric mixtures. Optically active (+) and (−), (R)- and (S)-, or (D)- and (L)-isomers may be prepared using chiral synthons or chiral reagents, or resolved using conventional techniques, such as HPLC. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column.

When the compounds described herein contain olefinic double bonds or other centers of geometric asymmetry, and unless specified otherwise, it is intended that the compounds include both E and Z geometric isomers. Likewise, all tautomeric forms are also intended to be included.

Compounds described herein also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

Compounds described herein also include pharmaceutically acceptable forms of the recited compounds, including chelates, non-covalent complexes, prodrugs, and mixtures thereof.

Compounds described herein also include different enriched isotopic forms, e.g., compounds enriched in the content of ²H, ³H, ¹¹C, ¹³C and/or ¹⁴C. In some embodiments, the compounds are deuterated. Such deuterated forms can be made by the procedure described in U.S. Pat. Nos. 5,846,514 and 6,334,997. As described in U.S. Pat. Nos. 5,846,514 and 6,334,997, deuteration may improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp. CAN 133:68895 AN 2000:473538 CAPLUS; Kabalka, George W.; Varma, Rajender S. The Synthesis of Radiolabeled Compounds via Organometallic Intermediates, Tetrahedron, 1989, 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem., 1981, 64(1-2), 9-32. CODEN: JRACBN ISSN:0022-4081, CAN 95:76229 AN 1981:476229 CAPLUS.

Acids (and bases) which are generally considered suitable for the formation of pharmaceutically acceptable salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Depending on its structure, the phrase “pharmaceutically acceptable salt,” as used herein, refers to a pharmaceutically acceptable organic or inorganic acid or base salt. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. Furthermore, a pharmaceutically acceptable salt can have more than one charged atom in its structure. In this instance the pharmaceutically acceptable salt can have multiple counterions. Hence, a pharmaceutically acceptable salt can have one or more charged atoms and/or one or more counterions.

Further, pharmaceutically acceptable salts include, but are not limited to aluminum, ammonium, calcium, copper, ferric, ferrous, lithium, magnesium, manganic, manganous, potassium, sodium, and zinc salts as well as salts derived from pharmaceutically acceptable organic non-toxic bases, such as salts of primary, secondary, and tertiary amines, substituted amines including naturally occurring substituted amines, cyclic amines, and basic ion exchange resins, e.g., arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

In addition, if the compound described herein is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, compounds also include prodrugs, for example ester or amide derivatives of the compounds described herein. As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The term “prodrugs”, as the term is used herein, is also intended to include any covalently bonded carriers which release an active parent drug in vivo when such prodrug is administered to a patient. Since prodrugs are known to enhance numerous desirable qualities of pharmaceuticals (i.e., solubility, bioavailability, manufacturing, etc.) the compounds or pharmaceutically acceptable salts described herein may be delivered in prodrug form. Thus, the skilled artisan will appreciate that the compounds or pharmaceutically acceptable salts described herein encompasses prodrugs, methods of delivering the same, and compositions containing the same. Prodrugs may be prepared by modifying functional groups present in the compound in such a way that the modifications are cleaved, either in routine manipulation or in vivo, to form the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs include compounds or pharmaceutically acceptable salts wherein a hydroxy, amino, or sulfhydryl group is bonded to any group that, when the prodrug is administered to a patient, it cleaves to form a free hydroxyl, free amino, or free sulfydryl group, respectively. Functional groups which may be rapidly transformed, by metabolic cleavage, in vivo form a class of groups reactive with the carboxyl group of the compounds or pharmaceutically acceptable salts described herein. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds or pharmaceutically acceptable salts described herein are cleaved in vivo, the compounds bearing such groups can act as prodrugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier, 1985; Methods in Enzymology, K. Widder et al, Ed., Academic Press, 42, p. 309 396, 1985; A Textbook of Drug Design and Development, Krogsgaard-Larsen and H. Bundgaard, ed., Chapter 5; “Design and Applications of Prodrugs” p. 113 191, 1991; Advanced Drug Delivery Reviews, H. Bundgard, 8, p. 138, 1992; Journal of Pharmaceutical Sciences, 77, p. 285, 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987; Bundgaard, H., Advanced Drug Delivery Review, 1992, 8, 1 38.

The term “solvate” is formed by the interaction of a solvent and a compound or pharmaceutically acceptable salt thereof. The term “compound” is intended to include solvates of compounds. Similarly, “salts” includes solvates of salts. Suitable solvates are pharmaceutically acceptable solvates, such as hydrates, including monohydrates and hemi-hydrates.

A “chelate” is formed by the coordination of a compound to a metal ion at two (or more) points. The term “compound” is intended to include chelates of compounds. Similarly, “salts” includes chelates of salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound’.

The term “hydrogen bond” refers to a form of association between an electronegative atom (also known as a hydrogen bond acceptor) and a hydrogen atom attached to a second, relatively electronegative atom (also known as a hydrogen bond donor). Suitable hydrogen bond donor and acceptors are well understood in medicinal chemistry (G. C. Pimentel and A. L. McClellan, The Hydrogen Bond, Freeman, San Francisco, 1960; R. Taylor and O. Kennard, “Hydrogen Bond Geometry in Organic Crystals”, Accounts of Chemical Research, 17, pp. 320-326 (1984)).

As used herein the terms “group”, “radical” or “fragment” are synonymous and are intended to indicate functional groups or fragments of molecules attachable to a bond or other fragments of molecules.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

The term “solution” means a mixture of one or more solutes in one or more solvents. Solution is intended to encompass homogeneous mixtures as well as heterogeneous mixtures, such as slurries or other mixtures having a suspension of insoluble (not dissolved) material.

As used herein, “alkyl”, “alkenyl” and “alkynyl” are straight or branched hydrocarbon chains, and if not specified, contain from 1 to 20 carbons or 2 to 20 carbons, such as from 1 to 16 carbons or 2 to 16 carbons. Alkenyl carbon chains having 2 to 20 carbons, in certain embodiments, contain 1 to 8 double bonds and alkenyl carbon chains having 2 to 16 carbons, in certain embodiments, contain 1 to 5 double bonds. Alkynyl carbon chains having 2 to 20 carbons, in certain embodiments, contain 1 to 8 triple bonds, and alkynyl carbon chains having 2 to 16 carbons, in certain embodiments, contain 1 to 5 triple bonds. Alkyl, alkenyl and alkynyl groups may be optionally substituted as described herein. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from 1 to 6 carbons.

As used herein, “alkylene” refers to a straight, branched or cyclic divalent aliphatic hydrocarbon group wherein the alkylene is attached to the rest of the molecule through two different bonds in the alkylene. In some embodiments the alkylene has from 1 to 20 carbon atoms, in another embodiment the alkylene has from 1 to 12 carbons. Alkylene groups may be optionally substituted as described herein. The term “lower alkylene” refers to alkylene groups having 1 to 6 carbons. In certain embodiments, alkylene groups are lower alkylene, including alkylene of 1 to 3 carbon atoms.

As used herein, “alkoxy” refers to an alkyl group attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Alkoxy groups will usually have from 1 to 6 carbon atoms attached through the oxygen bridge. The alkyl portion of alkoxy groups may be optionally substituted as described herein. “Lower alkoxy” refers to alkoxy groups having 1 to 6 (e.g., 1 to 4) carbons.

As used herein, “aralkyl” refers to a radical of the formula —R^{a}R^{d} where R^{a} is an alkylene radical as defined above, substituted by R^{d}, an aryl radical, as defined herein, e.g., benzyl. The alkylene and aryl radicals independently may be optionally substituted as described herein.

As used herein, “aryl” refers to aromatic monocyclic or multicyclic ring system containing from 6 to 14 carbon atoms. Aryl groups include, but are not limited to groups such as unsubstituted or substituted phenyl and unsubstituted or substituted naphthyl. Aryl groups may be optionally substituted as described herein.

As used herein, “cycloalkyl” refers to a saturated mono- or multi-cyclic ring system, in certain embodiments of 3 to 10 carbon atoms, in other embodiments of 3 to 6 carbon atoms. Cycloalkyl groups include multicyclic ring systems containing from 7 to 14 carbon atoms, where at least one ring is aromatic and at least one ring is partially or fully saturated (e.g., unsubstituted or substituted fluorenyl). Cycloalkyl groups also include mono- or multicyclic ring systems that respectively include at least one double bond and at least one triple bond (i.e., cycloalkenyl and cycloalkynyl). Cycloalkenyl groups may contain 3 to 10 carbon atoms, or 4 to 7 carbon atoms. Cycloalkynyl groups may contain 3 to 10 carbon atoms, or 8 to 10 carbon atoms. The ring systems of the cycloalkyl groups may be composed of one ring or two or more rings which may be joined together in a fused, bridged or spiro-connected fashion. Cycloalkyl groups may be optionally substituted as described herein.

As used herein, “cycloalkylalkyl” refers to a radical of the formula —R^{a}R^{b} where R^{a} is an alkylene radical as defined above and R^{b} is a cycloalkyl radical as defined above. The alkylene radical and the cycloalkyl radical independently may be optionally substituted as defined above.

As used herein, “heteroaralkyl” refers to a radical of the formula —R^{a}R^{e} where R^{a} is an alkylene radical as defined above and R^{e} is a heteroaryl radical as defined herein. The alkylene radical and the heteroaryl radical independently may be optionally substituted as defined herein.

As used herein, “heteroaryl” refers to a monocyclic or multicyclic aromatic heterocyclyl group, as defined herein, in certain embodiments, of about 5 to about 15 members where one or more, (e.g., 1 to 3) of the atoms in the ring system is a heteroatom selected from nitrogen, oxygen and sulfur. The heteroaryl group may be optionally fused to a benzene ring. Heteroaryl groups may be optionally substituted as defined herein. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, acridinyl, benzimidazolyl, benzothiazolyl, benzindolyl, benzothiadiazolyl, benzonaphthofuranyl, benzoxazolyl, benzofuranyl, benzothiophenyl, benzotriazolyl, benzo[4,6]imidazo[1,2-a]pyridinyl; carbazolyl, cinnolinyl, dibenzofuranyl, indolyl, indazolyl, isoindolyl, indolizinyl, naphthyridinyl, phenazinyl, phenothiazinyl, phenoxazinyl, phthalazinyl, pteridinyl, purinyll, quinazolinyl, quinoxalinyl, quinolinyl, and isoquinolinyl.

As used herein, “heterocyclyl” refers to a stable 3- to 18-membered ring system which consists of carbon atoms and from one to five heteroatoms selected from the group consisting of nitrogen, oxygen and sulfur. The heterocyclyl radical may be a monocyclic, bicyclic, tricyclic or tetracyclic ring system, which may include fused or bridged ring systems; the nitrogen, carbon or sulfur atoms in the heterocyclyl radical may be optionally oxidized; the nitrogen atom may be optionally quaternized; and the ring radical may be aromatic or partially or fully saturated. Heterocyclyl groups may be optionally substituted as defined herein. Examples of such heterocyclyl radicals include, but are not limited to, azepinyl, benzodioxolyl, benzodioxinyl, benzopyranyl, benzopyranonyl, benzofuranonyl, dioxolanyl, decahydroisoquinolyl, furanonyl. imidazolinyl, imidazolidinyl, isothiazolidinyl, indolinyl, isoindolinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, quinuclidinyl, thiazolidinyl, tetrahydrofuryl, tetrahydropyranyl, thiamorpholinyl, thiamorpholinyl sulfoxide, and thiamorpholinyl sulfone.

As used herein, “heterocyclylalkyl” refers to a radical of the formula —R^{a}R^{c} where R^{a} is an alkylene radical as defined above and R^{c} is a heterocyclyl radical as defined herein. The alkylene radical and the heterocyclyl radical independently may be optionally substituted as defined herein.

As used herein, “halo”, “halogen” or “halide” refers to F, Cl, Br or I.

As used herein, “haloalkyl” refers to an alkyl group in which one or more of the hydrogen atoms are replaced by halogen. Haloalkyl includes alkyl groups wherein all of the hydrogen atoms are replaced by halogen, i.e., perhaloalkyl. When the haloalkyl group contains more than one halogen, the halogens may be the same (e.g., dichloromethyl, trifluoromethyl) or different (e.g., 1-chloro-2-fluoroethyl). Haloalkyl groups include, but are not limited to, chloromethyl, trifluoromethyl and 1-chloro-2-fluoroethyl.

As used herein, “hydrazone” refers to a divalent group such as ═NNR^{t} which is attached to a carbon atom of another group, forming a double bond, wherein R^{t} is hydrogen or alkyl, as described herein.

As used herein, “imino” refers to a divalent group such as ═NR, which is attached to a carbon atom of another group, forming a double bond, wherein R is hydrogen or alkyl, as described herein.

Unless stated otherwise, optionally substituted alkyl, alkenyl and alkynyl refer to alkyl, alkenyl or alkynyl radicals, as defined herein, that may be optionally substituted by one or more (e.g., 1-6, 1-4, 1-2, or 1) substituents independently selected from nitro, halo, azido, cyano, cycloalkyl, aryl, heteroaryl, heterocyclyl, —ORₓ, —N(R_{y})(R_{z}), —SRₓ, —C(J)Rₓ, —C(J)ORₓ, —C(J)N(R_{y})(R_{z}), —C(J)SRₓ, —S(O)ₜRₓ (where t is 1 or 2), —OC(J)Rₓ, —OC(J)ORₓ, —OC(J)N(R_{y})(R_{z}), —OC(J)SRₓ, —N(Rₓ)C(J)Rₓ, —N(Rₓ)C(J)ORₓ, —N(Rₓ)C(J)N(R_{y})(R_{z}), —N(Rₓ)C(J)SRₓ, —Si(R_{w})₃, —N(Rₓ)S(O)₂R_{w}, —N(Rₓ)S(O)₂N(R_{y})(R_{z}), —S(O)₂N(R_{y})(R_{z}), —N(Rₓ)C(J)Rₓ, —P(O)(Rᵥ)₂, —OP(O)(Rᵥ)₂, —C(J)N(Rₓ)S(O)₂Rₓ, —C(J)N(Rₓ)N(Rₓ)S(O)₂Rₓ, —C(Rₓ)═N(ORₓ), and —C(Rₓ)═NN(R_{y})(R_{z}), wherein each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); and each J is independently O, NRₓ or S.

Unless stated otherwise, “optionally substituted aryl”, “optionally substituted cycloalkyl”, “optionally substituted heteroaryl”, and “optionally substituted heterocyclyl” refer to aryl, cycloalkyl, heterocyclyl, and heteroaryl radicals, respectively, as defined herein, that are optionally substituted by one or more (e.g., 1-6, 1-4, 1-2, or 1) substituents independently selected from nitro, halo, azido, cyano, alkyl, haloalkyl, alkenyl, alkynyl, cycloalkyl, cycloalkylalkyl, heterocyclyl, heterocyclylalkyl, aryl, aralkyl, heteroaryl, heteroaralkyl, —Rᵤ—ORₓ, —Rᵤ—N(R_{y})(R_{z}), —Rᵤ—SRₓ, —Rᵤ—C(J)Rₓ, —Rᵤ—C(J)ORₓ, —Rᵤ—C(J)N(R_{y})(R_{z}), —Rᵤ—C(J)SRₓ, —Rᵤ—S(O)ₜRₓ (where t is 1 or 2), —Rᵤ—OC(J)Rₓ, —Rᵤ—OC(J)Rₓ, —Rᵤ—OC(J)N(R_{y})(R_{z}), —RᵤOC(J)SRₓ, —Rᵤ—N(Rₓ)C(J)Rₓ, —Rᵤ—N(Rₓ)C(J)ORₓ, —Rᵤ—N(Rₓ)C(J)N(R_{y})(R_{z}), —Rᵤ—N(Rₓ)C(J)SRₓ, —Rᵤ—Si(R_{w})₃, —Rᵤ—N(Rₓ)S(O)₂R_{w}, —Rᵤ—N(Rₓ)S(O)₂N(R_{y})(R_{z}), —Rᵤ—S(O)₂N(R_{y})(R_{z}), —Rᵤ—N(Rₓ)C(J)Rₓ, —Rᵤ—P(O)(Rᵥ)₂, —Rᵤ—OP(O)(Rᵥ)₂, —Rᵤ—C(J)N(Rₓ)S(O)₂Rₓ, —Rᵤ—C(J)N(Rₓ)N(Rₓ)S(O)₂Rₓ, —Rᵤ—C(Rₓ)═N(ORₓ), and —Rᵤ—C(Rₓ)═NN(R_{y})(R_{z}), wherein each Rᵤ is independently alkylene or a direct bond; each Rᵥ is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORₓ or —N(R_{y})(R_{z}); each R_{w} is independently alkyl, alkenyl, alkynyl, cycloalkyl, cycloalkylalkyl, heterocyclyl, heterocyclylalkyl, aryl, aralkyl, heteroaryl, or heteroaralkyl; each Rₓ is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; R_{y} and R_{z} are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or R_{y} and R_{z}, together with the nitrogen atom to which they are attached, form a heterocyclyl or heteroaryl; and each J is O, NRₓ or S.

In some embodiments, optionally substituted alkyl and alkoxy refers to alkyl and alkoxy radicals, respectively, as defined herein, that are optionally substituted by one, two, or three substituents independently selected from halo, hydroxy, C₁-C₆ alkoxy, amino, C₁-C₆ alkylamino, and C₁-C₆ dialkylamino. In some embodiments, optionally substituted alkyl and alkoxy refers to alkyl and alkoxyo radicals, respectively, as defined herein, that are optionally substituted by one, two, or three substituents independently selected from halo, hydroxyl and C₁-C₆ alkoxy.

In some embodiments, optionally substituted aryl, cycloalkyl, heteroaryl, and heterocycloalkyl refers to aryl, cycloalkyl, heteroaryl, and heterocycloalkyl radicals, respectively, as defined herein, that are optionally substituted by one, two, or three substituents independently selected from C₁-C₆ alkyl, halo, hydroxy, C₁-C₆ alkoxy, amino, C₁-C₆ alkylamino, C₁-C₆ dialkylamino, heterocycloalkyl, —C(O)NH₂, —C(O)NH(C₁-C₆ alkyl), and —C(O)N(C₁-C₆ alkyl)₂. In some embodiments, optionally substituted aryl, cycloalkyl, heteroaryl, and heterocycloalkyl refers to aryl, cycloalkyl, heteroaryl, and heterocycloalkyl radicals, respectively, as defined herein, that are optionally substituted by one, two, or three substituents independently selected from C₁-C₆ alkyl, halo, hydroxy, C₁-C₆ alkoxy, heterocycloalkyl, —C(O)NH₂, —C(O)NH(C₁-C₆ alkyl), and —C(O)N(C₁-C₆ alkyl)₂.

Unless stated otherwise specifically in the specification, it is understood that the substitution can occur on any atom of the aryl, aralkyl, cycloalkyl, heterocyclyl, and heteroaryl groups.

Optionally substituted cycloalkyl and optionally substituted heterocyclyl may additionally be substituted with oxo, thioxo, imino, oxime or hydrazone, on a saturated carbon of their respective ring system.

As used herein, “oxime” refers to a divalent group such as ═N—OH, which is attached to a carbon atom of another group, forming a double bond.

As used herein, “oxo” refers to an oxygen atom doubly bonded to a carbon.

As used herein, “thioxo” refers to a sulfur atom doubly bonded to a carbon.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds are, unless indicated otherwise, in accord with their common usage, recognized abbreviations, or the IUPAC-IUB Commission on Biochemical Nomenclature (see, (1972) Biochem. 11:942-944).

If employed herein, the following terms have their accepted meaning in the chemical literature.
AcOH acetic acid
Boc tert-butoxycarbonyl
BOP benzotriazol-1-yloxy-tris(dimethylamino)-phosphonium hexafluorophosphate
DEAD diethyl azadicarboxylate
DMAP 4-(dimethylamino) pyridine
DMF N,N-dimethylformamide
DMSO dimethylsulf oxide
EDC 1-(3-dimethylaminopropyl)-3-ethylcarbodiimide hydrochloride
g gram or grams
h hour or hours
HATU O-(7-azabenzotriazol-1-yl)-N,N,N′,N′-tetramethyluronium hexafluorophosphate
HOBt 1-hydroxybenzotriazole hydrate
M molar
min or mins minute or minutes
ml or mL milliliter or milliliters
mmol millimole or millimoles
mol mole or moles
N normal
NMP 1-methyl-2-pyrrolidinone
psi pounds per square inch
TFA trifluoroacetic acid
THF tetrahydrofuran
TPP triphenylphosphine

Provided herein is a compound of Formula I

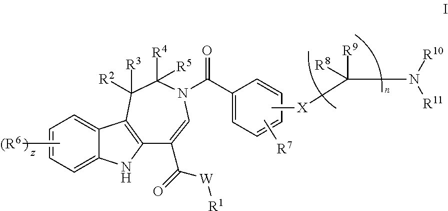

or a pharmaceutically acceptable salt thereof, wherein
W is chosen from O and NH;
X is chosen from O and CR⁸R⁹;
n is 2, 3 or 4 when X is equal to O, or
n is 0, 1, 2, 3 or 4 when X is equal to CR⁸R⁹;
y is 0, 1, or 2;
z is 1 or 2;
R¹ is chosen from optionally substituted alkyl, optionally substituted cycloalkyl, optionally substituted heterocyclyl, optionally substituted aryl, and optionally substituted heteroaryl;
R², R³, R⁴, and R⁵ are independently chosen from hydrogen and optionally substituted alkyl, or any two of R², R³, R⁴ and R⁵, together with the atoms to which they are attached, form an optionally substituted cycloalkyl or optionally substituted heterocyclyl ring;
R⁶, at each occurrence, independently is chosen from halogen, optionally substituted alkyl, hydroxyl, optionally substituted alkoxy and cyano;
R⁷ is chosen from hydrogen, halogen, optionally substituted alkyl, hydroxyl, optionally substituted alkoxy and cyano;
R⁸ and R⁹, at each occurrence, are independently chosen from hydrogen, fluoro, and alkyl; and
R¹⁰ and R¹¹ are independently chosen from hydrogen, optionally substituted alkyl, optionally substituted cycloalkyl and optionally substituted heterocyclyl, or R¹⁰ and R¹¹ together with the atoms to which they are attached, form an optionally substituted heterocyclyl ring containing 1 or 2 heteroatoms including the nitrogen through which they are attached.

In some embodiments, W is O.

In some embodiments, W is NH.

In some embodiments, the benzoyl group is meta- or para-substituted with —X—(CR⁸R⁹)ₙ—NR¹⁰R¹¹.

In some embodiments, X is O. In some embodiments when X is O, n is 2. In some embodiments when X is O, n is 3. In some embodiments when X is O, n is 4.

In some embodiments, X is C(R⁸R⁹). In some embodiments when X is C(R⁸R⁹), n is 0. In some embodiments when X is C(R⁸R⁹), n is 1. In some embodiments when X is C(R⁸R⁹)₂s, n is 2. In some embodiments when X is C(R⁸R⁹), n is 3. In some embodiments when X is C(R⁸R⁹), n is 4.

In some embodiments, z is 1. In some embodiments, z is 2.

In some embodiments, R¹ is optionally substituted alkyl. In some embodiments, R¹ is optionally substituted lower alkyl. In some embodiments, R¹ is lower alkyl. In some embodiments, R¹ is propyl. In some embodiments, R¹ is iso-propyl.

In some embodiments, R² and R³ are independently chosen from hydrogen and optionally substituted alkyl. In some embodiments, R² and R³ are independently chosen from hydrogen and optionally substituted lower alkyl. In some embodiments, R² and R³ are independently chosen from hydrogen and lower alkyl. In some embodiments, one of R² and R³ is hydrogen and the other is lower alkyl. In some embodiments, R² and R³ are each lower alkyl. In some embodiments, R² and R³ are each methyl.

In some embodiments, R⁴ and R⁵ are independently chosen from hydrogen and optionally substituted alkyl. In some embodiments, R⁴ and R⁵ are independently chosen from hydrogen and optionally substituted lower alkyl. In some embodiments, R⁴ and R⁵ are independently chosen from hydrogen and lower alkyl. In some embodiments, one of R⁴ and R⁵ is hydrogen and the other is lower alkyl. In some embodiments, R⁴ and R⁵ are each hydrogen.

In some embodiments, R⁶ is chosen from halogen and cyano. In some embodiments, R⁶ is halogen. In some embodiments, R⁶ is fluoro.

In some embodiments, R⁷ is chosen from hydrogen, halogen, optionally substituted lower alkyl, hydroxyl, optionally substituted lower alkoxy and cyano. In some embodiments, R⁷ is chosen from hydrogen, halogen, lower alkyl, lower alkoxy, and cyano. In some embodiments, R⁷ is chosen from hydrogen and halogen. In some embodiments, R⁷ is hydrogen.

In some embodiments, for each occurrence, R⁸ and R⁹ are independently chosen from hydrogen, fluoro, and lower alkyl. In some embodiments, for each occurrence, R⁸ and R⁹ are independently chosen from hydrogen and lower alkyl. In some embodiments, for each occurrence, one of R⁸ and R⁹ is hydrogen and the other is lower alkyl. In some embodiments, for each occurrence, R⁸ and R⁹ are hydrogen.

In some embodiments, R¹⁰ and R¹¹ are independently chosen from hydrogen, optionally substituted lower alkyl, optionally substituted cycloalkyl and optionally substituted heterocyclyl. In some embodiments, R¹⁰ and R¹¹ are independently chosen from hydrogen and optionally substituted lower alkyl. In some embodiments, R¹⁰ and R¹¹ are independently chosen from hydrogen and lower alkyl. In some embodiments, R¹⁰ and R¹¹ are hydrogen.

In some embodiments, R¹⁰ and R¹¹ together with the atoms to which they are attached, form an optionally substituted 5- to 7-membered heterocyclyl ring containing 1 or 2 heteroatoms including the nitrogen through which they are attached. In some embodiments, R¹⁰ and R¹¹ together with the atoms to which they are attached, form an optionally substituted 5- to 7-membered heterocyclyl ring chosen from morpholinyl, piperidinyl, piperazinyl, 4-piperidonyl, pyrrolidinyl, pyrazolidinyl, quinuclidinyl, thiazolidinyl, tetrahydrofuryl, tetrahydropyranyl, thiamorpholinyl, thiamorpholinyl sulfoxide, and thiamorpholinyl sulfone, each of which is optionally substituted.

In some embodiments, R¹⁰ and R¹¹ together with the atoms to which they are attached, form an optionally substituted 5- to 7-membered heterocyclyl ring chosen from morpholinyl, piperidinyl, piperazinyl, 4-piperidonyl, pyrrolidinyl, pyrazolidinyl, thiamorpholinyl, thiamorpholinyl sulfoxide, and thiamorpholinyl sulfone, each of which is optionally substituted. In some embodiments, R¹⁰ and R¹¹ together with the atoms to which they are attached, form an optionally substituted 5- to 7-membered heterocyclyl ring chosen from morpholinyl, thiamorpholinyl, thiamorpholinyl sulfoxide, and thiamorpholinyl sulfone, each of which is optionally substituted. In some embodiments, R¹⁰ and R¹¹ together with the atoms to which they are attached, form a morpholinyl ring.

Also provided is a compound of Formula II

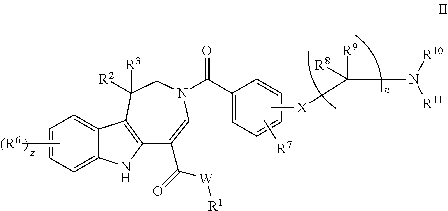

or a pharmaceutically acceptable salt thereof wherein n, R¹, R², R³, R⁶, R⁷, R⁸, R⁹, R¹⁰, R¹¹, W, and X are as described for compounds of Formula I.

Also provided is a compound of Formula III

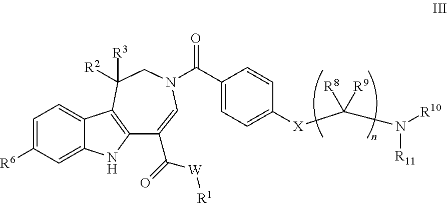

or a pharmaceutically acceptable salt thereof wherein n, R¹, R², R³, R⁶, R⁸, R⁹, R¹⁰, R¹¹, W, and X are as described for compounds of Formula I.

Also provided is a compound of Formula IV

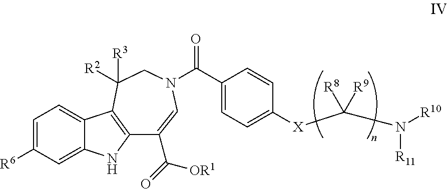

or a pharmaceutically acceptable salt thereof wherein n, R¹, R², R³, R⁶, R⁸, R⁹, R¹⁰, R¹¹, and X are as described for compounds of Formula I.

Also provided is a compound of Formula V

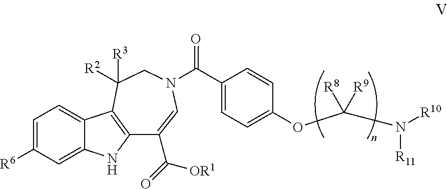

or a pharmaceutically acceptable salt thereof wherein n, R¹, R², R³, R⁶, R⁸, R⁹, R¹⁰, and R¹¹ are as described for compounds of Formula I.

Also provided is a compound of Formula VI

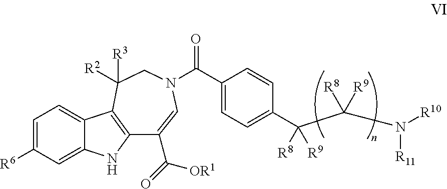

or a pharmaceutically acceptable salt thereof wherein n, R¹, R², R³, R⁶, R⁸, R⁹, R¹⁰, and R¹¹ are as described for compounds of Formula I.

In some embodiments, the compound of Formula I is chosen from:
isopropyl 8-fluoro-1,1-dimethyl-3-[4-(morpholin-4-ylmethyl)benzoyl]-1,2,3,6-tetrahydroazepino[4,5-b]indole-5-carboxylate;
isopropyl 8-fluoro-1,1-dimethyl-3-[4-(2-morpholin-4-ylethyl)benzoyl]-1,2,3,6-tetrahydroazepino[4,5-b]indole-5-carboxylate;
isopropyl 8-fluoro-1,1-dimethyl-3-[4-(3-morpholin-4-ylpropoxy)benzoyl]-1,2,3,6-tetrahydroazepino[4,5-b]indole-5-carboxylate; and
isopropyl 8-fluoro-1,1-dimethyl-3-[4-(2-morpholin-4-ylethoxy)benzoyl]-1,2,3,6-tetrahydroazepino[4,5-b]indole-5-carboxylate and pharmaceutically acceptable salts, solvates, and prodrugs thereof.

Also provided is at least one compound chosen from:
isopropyl 1,1-dimethyl-3-[4-(3-pyrrolidin-1-ylpropoxy)benzoyl]-1,2,3,6-tetrahydroazepino[4,5-b]indole-5-carboxylate;
isopropyl 1,1-dimethyl-3-{4-[2-(4-methylpiperazin-1-yl)ethoxy]benzoyl}-1,2,3,6-tetrahydroazepino[4,5-b]indole-5-carboxylate;
isopropyl 1,1-dimethyl-3-{4-[3-(4-methylpiperazin-1-yl)propoxy]benzoyl}-1,2,3,6-tetrahydroazepino[4,5-b]indole-5-carboxylate;
isopropyl 1,1-dimethyl-3-[3-(2-pyrrolidin-1-ylethoxy)benzoyl]-1,2,3,6-tetrahydroazepino[4,5-b]indole-5-carboxylate;
isopropyl 1,1-dimethyl-3-{3-[2-(4-methylpiperazin-1-yl)ethoxy]benzoyl}-1,2,3,6-tetrahydroazepino[4,5-b]indole-5-carboxylate;
isopropyl 1,1-dimethyl-3-[3-(3-pyrrolidin-1-ylpropoxy)benzoyl]-1,2,3,6-tetrahydroazepino[4,5-b]indole-5-carboxylate;
isopropyl 1,1-dimethyl-3-{3-[3-(4-methylpiperazin-1-yl)propoxy]benzoyl}-1,2,3,6-tetrahydroazepino[4,5-b]indole-5-carboxylate;
isopropyl 1,1-dimethyl-3-[4-(morpholin-4-ylmethyl)benzoyl]-1,2,3,6-tetrahydroazepino[4,5-b]indole-5-carboxylate;
isopropyl 1,1-dimethyl-3-[4-(2-morpholin-4-ylethyl)benzoyl]-1,2,3,6-tetrahydroazepino[4,5-b]indole-5-carboxylate;
isopropyl 3-(3-{[(2R,6R)-2,6-dimethylmorpholin-4-yl]methyl}benzoyl)-1,1-dimethyl-1,2,3,6-tetrahydroazepino[4,5-b]indole-5-carboxylate;
isopropyl 3-(3-{[(2R,6S)-2,6-dimethylmorpholin-4-yl]methyl}benzoyl)-1,1-dimethyl-1,2,3,6-tetrahydroazepino[4,5-b]indole-5-carboxylate;
isopropyl 3-(3-{[(3R,5S)-3,5-dimethylpiperazin-1-yl]methy}benzoyl)-1,1-dimethyl-1,2,3,6-tetrahydroazepino[4,5-b]indole-5-carboxylate;
isopropyl 3-{3-[(4-carbamoylpiperidin-1-yl)methyl]benzoyl}-1,1-dimethyl-1,2,3,6-tetrahydroazepino[4,5-b]indole-5-carboxylate;
isopropyl 1,1-dimethyl-3-{3-[(4-morpholin-4-ylpiperidin-1-yl)methyl]benzoyl}-1,2,3,6-tetrahydroazepino[4,5-b]indole-5-carboxylate;
isopropyl 1,1-dimethyl-3-[3-(1,3-thiazolidin-3-ylmethyl)benzoyl]-1,2,3,6-tetrahydroazepino[4,5-b]indole-5-carboxylate;
isopropyl 3-[3-(1,4′-bipiperidin-1′-ylmethyl)benzoyl]-1,1-dimethyl-1,2,3,6-tetrahydroazepino[4,5-b]indole-5-carboxylate;
isopropyl 1,1-dimethyl-3-{3-[(4-pyrrolidin-1-ylpiperidin-1-yl)methyl]benzoyl}-1,2,3,6-tetrahydroazepino[4,5-b]indole-5-carboxylate;
isopropyl 3-(3-{[(3R,5S)-3,5-dimethylpiperidin-1-yl]methyl}benzoyl)-1,1-dimethyl-1,2,3,6-tetrahydroazepino[4,5-b]indole-5-carboxylate;
isopropyl 3-(3-{[(3S,5S)-3,5-dimethylpiperidin-1-yl]methyl}benzoyl)-1,1-dimethyl-1,2,3,6-tetrahydroazepino[4,5-b]indole-5-carboxylate;
isopropyl 1,1-dimethyl-3-[3-(thiomorpholin-4-ylmethyl)benzoyl]-1,2,3,6-tetrahydroazepino[4,5-b]indole-5-carboxylate;
isopropyl 3-(3-{[(2S,5S)-2,5-dimethylpyrrolidin-1-yl]methyl}benzoyl)-1,1-dimethyl-1,2,3,6-tetrahydroazepino[4,5-b]indole-5-carboxylate;
isopropyl 3-{3-[(cyclohexylamino)methyl]benzoyl}-1,1-dimethyl-1,2,3,6-tetrahydroazepino[4,5-b]indole-5-carboxylate;
isopropyl 3-(3-{[cyclohexyl(methyl)amino]methyl)benzoyl}-1,1-dimethyl-1,2,3,6-tetrahydroazepino[4,5-b]indole-5-carboxylate;
isopropyl 3-{3-[(4-hydroxypiperidin-1-yl)methyl]benzoyl}-1,1-dimethyl-1,2,3,6-tetrahydroazepino[4,5-b]indole-5-carboxylate;
isopropyl 3-{3-[(3-carbamoylpiperidin-1-yl)methyl]benzoyl}-1,1-dimethyl-1,2,3,6-tetrahydroazepino[4,5-b]indole-5-carboxylate;
isopropyl 1,1-dimethyl-3-{3-[(piperidin-4-ylamino)methyl]benzoyl}-1,2,3,6-tetrahydroazepino[4,5-b]indole-5-carboxylate; and
isopropyl 3-{3-[(1,1-dioxidothiomorpholin-4-yl)methyl]benzoyl}-1,1-dimethyl-1,2,3,6-tetrahydroazepino[4,5-b]indole-5-carboxylate,
   or a pharmaceutically acceptable salt thereof.

The reagents used in the preparation of the compounds described herein can be either commercially obtained or can be prepared by standard procedures described in the literature (e.g., March's Advanced Organic Chemistry: Reactions, Mechanisms, and Structure, (2007) 6th Ed.; Wiley-Interscience, New York). Protecting groups may be added or removed in accordance with standard techniques, which are well-known to those skilled in the art and as described herein. The use of protecting groups is described in detail in Greene, T. W. and Wuts, P. G. M. Greene's Protective Groups in Organic Synthesis (2007), 4th Ed., Wiley-Interscience. The preparation of compounds of formulas 2-7, have been disclosed previously (WO2003099821 and WO2005009387). In some embodiments, compounds described herein are produced by the following reaction schemes.

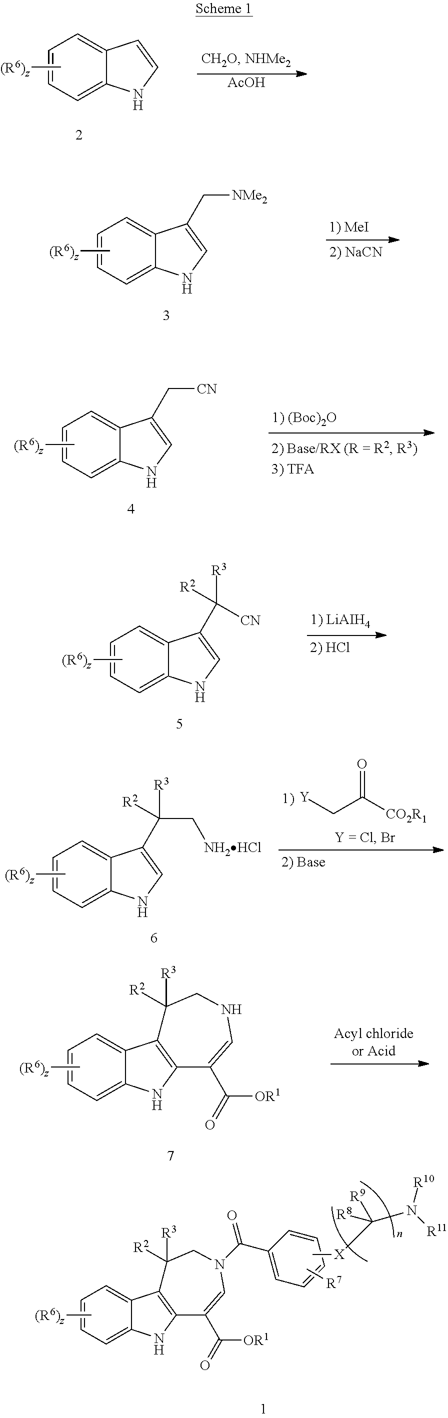

As depicted in Scheme 1, compounds of formula I can be prepared from compounds of formula 7 via acylation. Acylation of the amine can be achieved by any conventional method for the formation of a peptide bond including but not limited to: 1) treatment of compounds of formula 7 with a carboxylic acid and a coupling agent including but not limited to: HATU, BOP, EDC/DMAP, and EDC/HOBt; and 2) treatment of compounds of formula 7 with base and an acyl chloride. In some embodiments, the azepine is treated with the appropriate acyl chloride in the presence of a base, such as triethylamine.

Compounds of formula 7 can be prepared from compounds of formula 6 via cyclization followed by a rearrangement reaction. Any conventional method to form the appropriate azepine ring can be employed. In some embodiments, compounds of formula 6 are treated with the appropriately substituted halo-pyruvate, either as the bromopyruvate, chloropyruvate, or a mixture of the two and heated at 80° C. Upon completion of the initial cyclization, pyridine and DMAP are added and heated at 80° C. to effect rearrangement to the azepine.

Compounds of formula 6 are prepared from compounds of formula 5 by reduction followed by salt formation. Any conventional method for the reduction of a nitrile to an amine and any conventional method for the formation of a salt of a basic amine can be employed. In some embodiments, the nitrile is reduced (e.g., with lithium aluminum hydride) or hydrogenated (e.g., at 30-60 psi in a Parr apparatus in the presence of Raney nickel). Formation of the salt is accomplished by treatment with acid (e.g. HCl).

Compounds of formula 5 are formed from compounds of formula 4 by protection of the indole nitrogen, followed by alkylation, followed by deprotection. Any conventional methods and protecting groups used to block reaction of an indole nitrogen can be employed. In addition, any method for alkylation of 3-indolylacetonitriles followed by any conventional method for the deprotection of an indole nitrogen can be used for this transformation. In some embodiments, the indole nitrogen in compounds of formula 4 is protected with the Boc (tert-butoxycarbonyl) group. This transformation is performed by reacting compounds of formula 4 with Boc anhydride in the presence of a base such as triethylamine and a coupling catalyst such as DMAP. Alkylation of the N-protected 3-indolylacetonitrile is accomplished by forming the anion with a base, such as sodium hydride and reacting it with an alkylating agent such as an alkylhalide, such as an alkylbromide or alkyliodide. If it is desired to form a dialkyl analog, then two equivalents of base and alkylating agent can be employed. Finally, deprotection of the indole nitrogen is accomplished in the presence of an acid such as trifluoroacetic acid.

Intermediates 4 can be prepared readily from gramines 3, which are either commercially available or synthesized from indoles 2 (Brown and Garrison, J. Am. Chem. Soc. 1955, 77, 3839-3842). In general, gramines 3 can be treated with methyl iodide to form a quaternary ammonium salt, which can be displaced with cyanide to give 3-indolylacetonitriles 4.

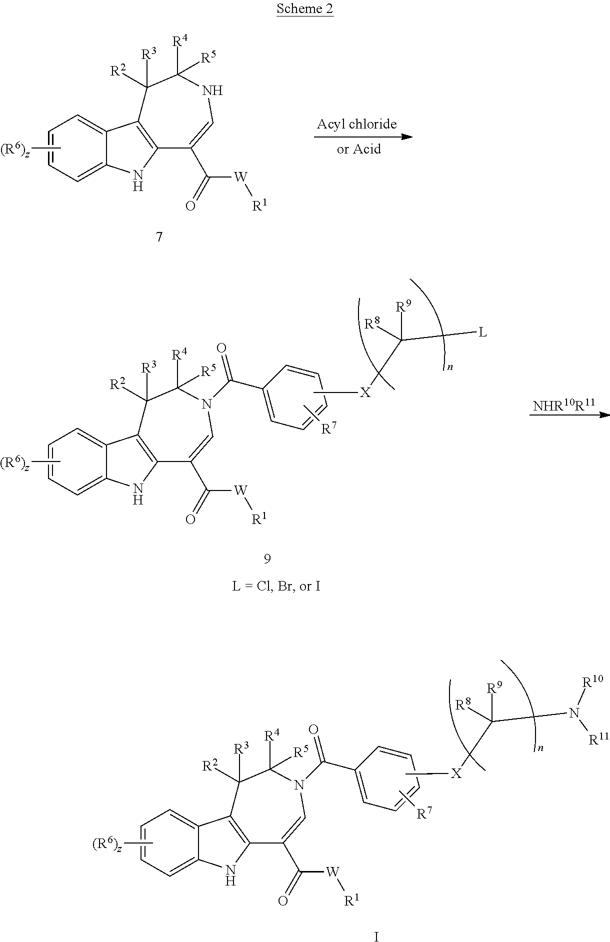

As depicted in Scheme 2, Compounds of Formula I can be formed through preparation of a common intermediate 9, containing a leaving group L, which can be displaced with the requisite amine. Compounds of formula 9 can be prepared from compounds of formula 7 via acylation. Acylation of the amine can be achieved by any conventional method for the formation of a peptide bond including but not limited to: 1) treatment of compounds of formula 7 with a carboxylic acid and a coupling agent including but not limited to: HATU, BOP, EDC/DMAP, EDC/triethylamine, and EDC/HOBt; and 2) treatment of compounds of formula 7 with base and an acyl chloride. In some embodiments, the azepine is treated with the appropriate acyl chloride in the presence of a base, such as triethylamine.

Any conventional means for displacing a leaving group (L) with an amine can be employed for this transformation. In some embodiments, compounds of formula 9, such as where L is equal to Cl, Br, or I, are treated with excess amine to form compounds of formula I.

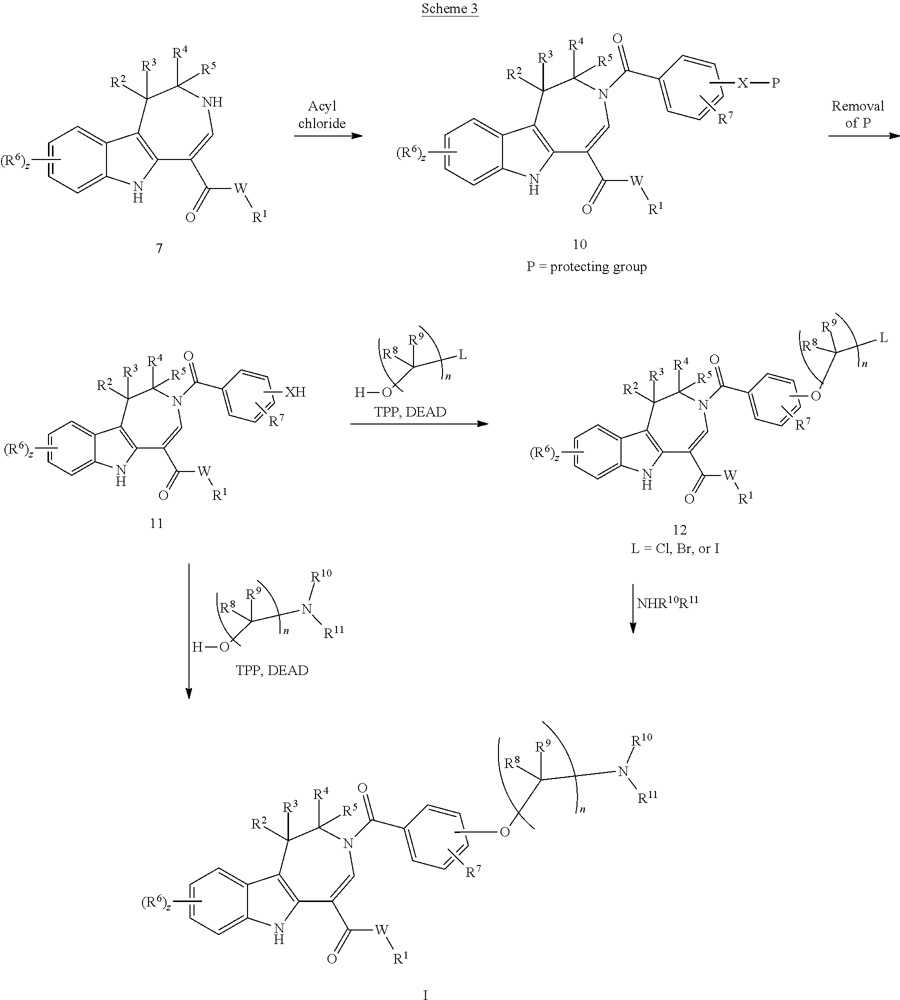

Compounds of formula I where X=O, and R¹, R², R³, R⁴, R⁵, R⁶, R⁷, R⁸, R⁹, R¹⁰, R¹¹, Z, and n are as previously described can be prepared from phenols (11, X=O) as outlined in Scheme 3. This transformation can be carried out using any conventional method for preparation of phenyl ethers including but not limited to: 1) treatment of compound 11 with the requisite amino alcohol using Mitsunobu conditions to provide targets I directly; and 2) treatment of compound 11 with the requisite alcohol containing a pendant leaving group such as a halo functionality using Mitsunobu conditions to produce compounds of formula 12. The halo-functionality in 12 can be converted to compounds of formula I via nucleophilic substitution with the appropriately substituted amine. Phenols of formula 11 (X=O) are prepared from compounds of formula 7 by acylation with an acid halide formed from an appropriately substituted and protected hydroxybenzoic acid. Any conventional protecting group (P) for a phenol may be employed and any conventional reagent for converting an acid to an acid halide may be used. In some embodiments, P is benzyl, which can be removed using phase-transfer hydrogenolysis. Formation of the acid chloride can be accomplished with thionyl chloride at temperatures between room temperature and reflux, and coupling to the azepine can be accomplished as previously described.

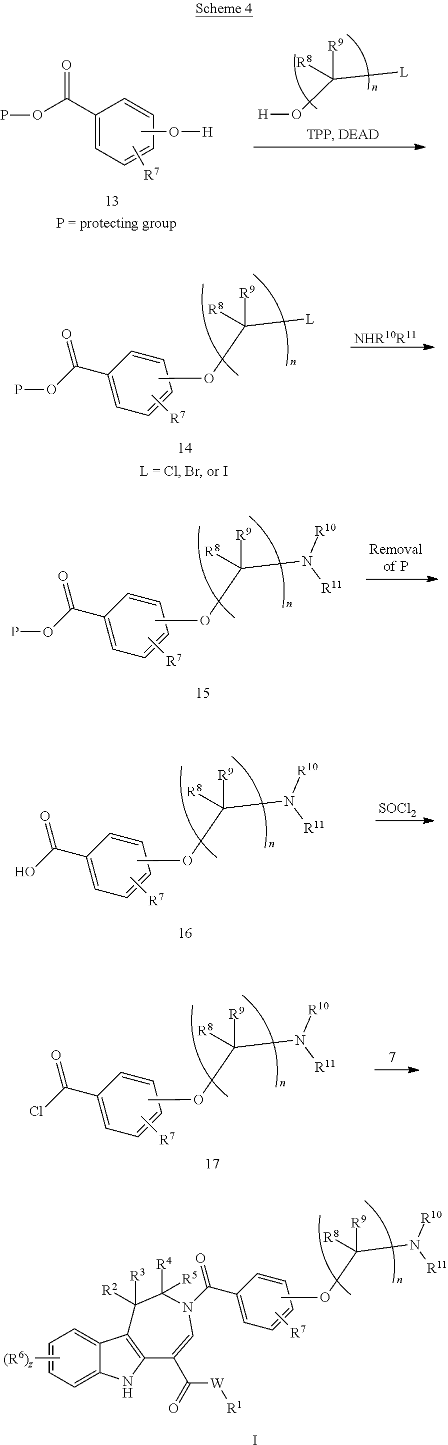

In some embodiments, compounds of Formula I where X=O can be prepared by treating azepines 7 with fully-elaborated benzoyl chlorides 17 as depicted in Scheme 4. Compounds 17, can be prepared by treating the amino acid 16 with thionyl chloride or any other method for converting an acid to an acid halide. The acid of formula 16 can be prepared from the appropriately substituted hydroxybenzoate of formula 13 by alkylation of the phenol, followed by displacement of the leaving group L with an amine, followed by liberation of the benzoic acid. Any suitable protecting group P may be employed and any conventional method for removing the protecting group may be used. In addition, any conventional method for alkylation of a phenol and any conventional method for displacing a leaving group with an amine may be employed. In some embodiments, P is benzyl. In some embodiments, compounds of formula 16 (X=O), are prepared by alkylating the phenol group of benzyl esters (13) using diethylazodicarboxylate, triphenylphosphine, and the desired halo-alkanol to provide compounds of formula 14. The halo-functionality is displaced by treating compounds of formula 14 with an excess amount of the desired amine to provide benzyl esters 15. The benzyl-protecting group is removed using phase-transfer hydrogenolysis (e.g., by treatment with excess 1,4-cyclohexadiene and catalytic palladium hydroxide on carbon at elevated temperatures, e.g. 40-80° C.) to provide amino acids 16.

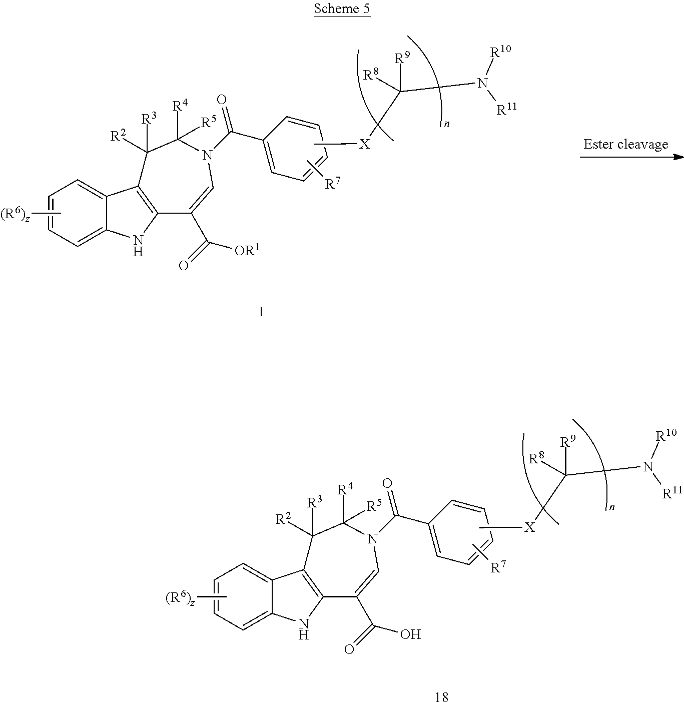

Compounds of formula 18 can be prepared by the hydrolysis or cleavage of compounds of formula I as depicted in Scheme 5. The conversion can be accomplished using any conventional method for hydrolysis or cleavage of an ester. In some embodiments, compounds of formula I where R¹ is equal to ethyl are treated with lithium chloride in DMF, and either irradiated in a microwave at 180° C. or heated at reflux.

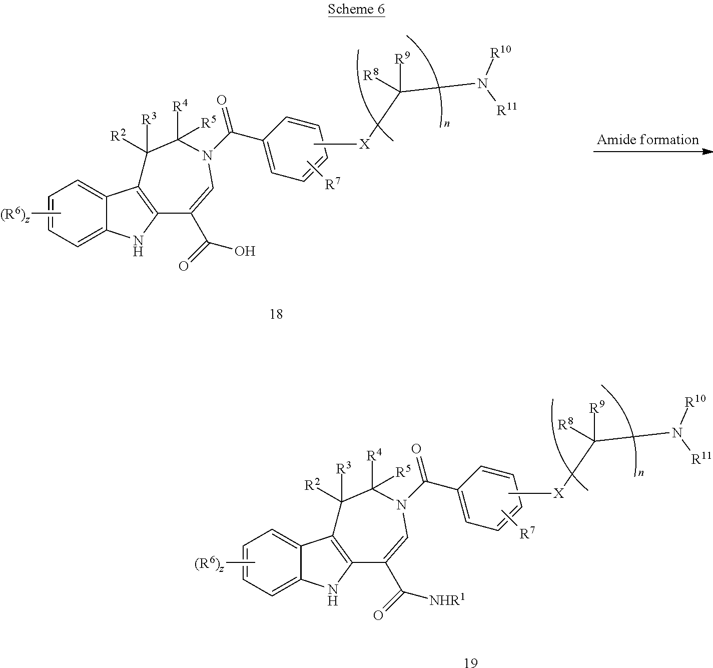

Amides of formula 19 can be prepared from carboxylic acids of formula 18 as depicted in Scheme 6. The conversion can be performed using any conventional acid activating reagent including, but not limited to: HATU, BOP, EDC/DMAP, and EDC/HOBt and treatment with amine. In some embodiments, compounds of formula 18 are treated with HATU and the requisite amine in NMP.

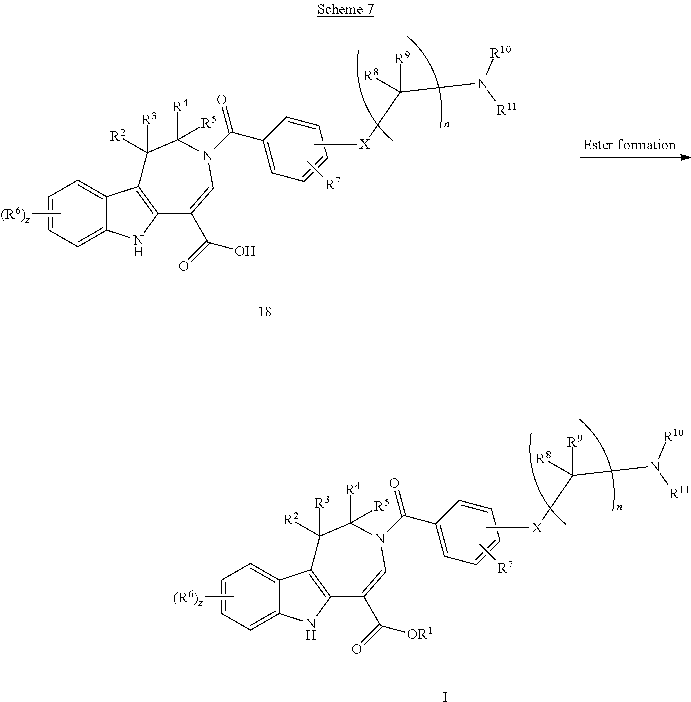

Esters of formula I can be prepared from carboxylic acids of formula 18 as depicted in Scheme 7. The conversion can be performed using any conventional acid activating reagent including, but not limited to: HATU, BOP, EDC/DMAP, and EDC/HOBt and treatment with alcohol. In some embodiments, compounds of formula 18 are treated with EDC for activation, followed by addition of the requisite alcohol and DMAP.

Also provided is a pharmaceutical composition comprising a therapeutically effective amount of at least one compound or pharmaceutically acceptable salt thereof described herein.

The compounds and pharmaceutically acceptable salts described herein, as a pharmaceutical composition, can be formulated neat or with a pharmaceutical carrier for administration, the proportion of which is determined by the solubility and chemical nature of the compound, chosen route of administration and standard pharmacological practice. The pharmaceutical carrier may be solid or liquid.

A solid carrier can include one or more substances which also may act as a flavoring agent, sweetening agent, lubricant, solubilizer, suspending agent, filler, glidant, compression aid, binder, or tablet-disintegrating agent; it also can be an encapsulating material. In powders, the carrier is a finely divided solid that is in admixture with the finely divided active ingredient.

Solid dosage unit forms or compositions such as tablets, troches, pills, capsules, powders, and the like, may contain a solid carrier binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose, or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both.

Liquid carriers are used in preparing liquid dosage forms such as solutions, suspensions, dispersions, emulsions, syrups, elixirs and pressurized compositions. The active ingredient can be dissolved or suspended in a pharmaceutically acceptable liquid carrier such as water, an organic solvent, a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g., cellulose derivatives, such as, sodium carboxymethyl cellulose solution); alcohols, including monohydric alcohols such as ethanol and polyhydric alcohols such as glycols and their derivatives; lethicins, and oils such as fractionated coconut oil and arachis oil. For parenteral administration, the liquid carrier also can be an oily ester such as ethyl oleate and isopropyl myristate. Sterile liquid carriers are useful in sterile liquid form compositions for parenteral administration. The liquid carrier for pressurized compositions can be a halogenated hydrocarbon or other pharmaceutically acceptable propellant.

A liquid pharmaceutical composition such as a syrup or elixir may contain, in addition to one or more liquid carriers and the active ingredients, a sweetening agent such as sucrose, preservatives such as methyl and propyl parabens, a pharmaceutically acceptable dye or coloring agent, or a flavoring agent such as cherry or orange flavoring.

Liquid pharmaceutical compositions that are sterile solutions or suspensions can be administered intraocularly or parenterally, for example, by intramuscular, intraperitoneal or subcutaneous injection. Sterile solutions also can be administered intravenously. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy injectability exists. It must be stable under the conditions of manufacture and storage and must be preserved against the contaminating action of microorganisms such as bacteria and fungi. The carrier can be a solvent or dispersion medium containing a liquid carrier, for example, water, ethanol, polyol (e.g., glycerol, propylene glycol and liquid polyethylene glycol), suitable mixtures thereof, and vegetable oils. The liquid carrier may be suitably mixed with a surfactant such as hydroxypropylcellulose.

The compounds described herein also may be administered rectally or vaginally in the form of a conventional suppository. For administration by intranasal or intrabronchial inhalation or insufflation, the compounds described herein may be formulated into an aqueous or partially aqueous solution, which can then be utilized in the form of an aerosol. The compounds described herein may be administered topically, or also transdermally through the use of a transdermal patch containing the active compound and a carrier that is inert to the active compound, which is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient also may be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

In some embodiments, the pharmaceutical composition is combined with one or more additional active ingredients selected from antihyperlipidemic agents, plasma HDL-raising agents, antihypercholesterolemic agents, cholesterol biosynthesis inhibitors, HMG CoA reductase inhibitors, acyl-coenzyme A cholesterol acytransferase (ACAT) inhibitors, probucol, raloxifene, nicotinic acid, niacinamide, cholesterol absorption inhibitors, bile acid sequestrants, low density lipoprotein receptor inducers, clofibrate, fenofibrate, benzofibrate, cipofibrate, gemfibrizol, vitamin B₆, vitamin B₁₂, vitamin C, vitamin E, βblockers, anti-diabetes agents, sulfonylureas, biguanides, thiazolidinediones, activators of PPARα PPARβ and PPARγ, dehydroepiandrosterone, antiglucocorticoids, TNF α inhibitors, α-glucosidase inhibitors, pramlintide, amylin, insulin, angiotensin II antagonists, angiotensin converting enzyme inhibitors, platelet aggregation inhibitors, fibrinogen receptor antagonists, LXR α agonists, antagonists or partial agonists, LXR β agonists, antagonists or partial agonists, phenylpropanolamine, phentermine, diethylpropion, mazindol, fenfluramine, dexfenfluramine, phentiramine, β₃ adrenoceptor agonist agents, sibutramine, gastrointestinal lipase inhibitors, neuropeptide Y, enterostatin, cholecytokinin, bombesin, amylin, histamine H₃ receptor agonists or antagonists, dopamine D₂ receptor agonists or antagonists, melanocyte stimulating hormone, corticotrophin releasing factor, leptins, galanin or gamma amino butyric acid (GABA), aspirin, or fibric acid derivatives.

Provided is a method of treating, preventing, inhibiting, or ameliorating one or more symptoms of a disease or disorder in which nuclear receptor activity is implicated, comprising administering to a subject in need thereof an effective amount of at least one compound or pharmaceutically acceptable salt thereof described herein or a composition comprising at least one compound or pharmaceutically acceptable salt thereof described herein.

In some embodiments, the nuclear receptor, for which activity is indicated, is the farnesoid X receptor.

In some embodiments, the composition comprising at least one compound or pharmaceutically acceptable salt thereof described herein further comprises one or more additional agents selected from antihyperlipidemic agents, plasma HDL-raising agents, antihypercholesterolemic agents, cholesterol biosynthesis inhibitors, HMG CoA reductase inhibitors, acyl-coenzyme A:cholesterol acytransferase (ACAT) inhibitors, probucol, raloxifene, nicotinic acid, niacinamide, cholesterol absorption inhibitors, bile acid sequestrants, low density lipoprotein receptor inducers, clofibrate, fenofibrate, benzofibrate, cipofibrate, gemfibrizol, vitamin B₆, vitamin B₁₂, vitamin C, vitamin E, β-blockers, anti-diabetes agents, sulfonylureas, biguanides, thiazolidinediones; activators of PPARα, PPARβ and PPARγ, dehydroepiandrosterone, antiglucocorticoids, TNFα inhibitors, α-glucosidase inhibitors, pramlintide, amylin, insulin, angiotensin II antagonists, angiotensin converting enzyme inhibitors, platelet aggregation inhibitors, fibrinogen receptor antagonists, LXR α agonists, partial agonists or antagonists, LXR β agonists, partial agonists or antagonists, phenylpropanolamine, phentermine, diethylpropion, mazindol, fenfluramine, dexfenfluramine, phentiramine, β₃ adrenoceptor agonist agents, sibutramine, gastrointestinal lipase inhibitors, neuropeptide Y, enterostatin, cholecytokinin, bombesin, amylin, histamine H₃ receptor agonists or antagonists, dopamine D₂ receptor agonists or antagonists, melanocyte stimulating hormone, corticotrophin releasing factor, leptin, galanin, gamma amino butyric acid (GABA), aspirin, or fibric acid derivatives, simultaneously with, prior to, or after administration of the compound.

In some embodiments, the compound is a farnesoid X receptor agonist, partial agonist, inverse agonist, partial antagonist, or antagonist.

In some embodiments, the disease or disorder is selected from hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, and obesity.

In some embodiments, the disease or disorder is hyperlipidemia.

In some embodiments, the disease or disorder is hypertriglyceridemia.

In some embodiments, the disease or disorder is hypercholesterolemia.

In some embodiments, the disease or disorder is obesity.

In some embodiments, the disease or disorder is cholestasis.

In some embodiments, the disease or disorder is atherosclerosis.

In some embodiments, the method further comprises administering at least one additional active agent selected from phenylpropanolamine, phentermine, diethylpropion, mazindol, fenfluramine, dexfenfluramine, phentiramine, β₃ adrenoceptor agonist agents, sibutramine, gastrointestinal lipase inhibitors, LXR α agonists, partial agonists or antagonists, LXR β agonists, partial agonists or antagonists, neuropeptide Y, enterostatin, cholecytokinin, bombesin, amylin, histamine H₃ receptor agonists or antagonists, dopamine D₂ receptor agonists or antagonists, melanocyte stimulating hormone, corticotrophin releasing factor, leptins, galanin, or gamma amino butyric acid (GABA) simultaneously with, prior to, or after administration of the compound.

In some embodiments, the disease or disorder is selected from hyperlipidemia, hypertriglyceridemia, hypercholesterolemia, and dyslipidemia.

In some embodiments, the method further comprises administering one or more additional active agents selected from antihyperlipidemic agents, plasma HDL-raising agents, antihypercholesterolemic agents, cholesterol biosynthesis inhibitors, HMG CoA reductase inhibitors, acyl-coenzyme A:cholesterol acytransferase (ACAT) inhibitors, probucol, raloxifene, nicotinic acid, niacinamide, cholesterol absorption inhibitors, bile acid sequestrants, low density lipoprotein receptor inducers, clofibrate, fenofibrate, benzofibrate, cipofibrate, gemfibrizol, vitamin B₆, vitamin B₁₂, anti-oxidant vitamins, β-blockers, anti-diabetes agents, angiotensin II antagonists, angiotensin converting enzyme inhibitors, platelet aggregation inhibitors, fibrinogen receptor antagonists, aspirin, LXR α agonists, partial agonists or antagonists, LXR β agonists, partial agonists or antagonists, or fibric acid derivatives, simultaneously with, prior to, or after administration of a compound or composition described herein.

In some embodiments, the disease or disorder is selected from atherosclerosis, atherosclerotic disease, atherosclerotic disease events, and atherosclerotic cardiovascular disease.

In some embodiments, the disease or disorder is selected from Syndrome X, diabetes mellitus, type II diabetes, insulin insensitivity, and hyperglycemia.

In some embodiments, the method further comprises administering at least one additional agent selected from sulfonylureas, biguanides, thiazolidinediones, activators of PPARα, PPARβ and PPARγ, agonists, LXR α agonists, partial agonists or antagonists, LXR β agonists, partial agonists or antagonists, dehydroepiandrosterone, antiglucocorticoids; TNF α inhibitors, A-glucosidase inhibitors, pramlintide, amylin, insulin or insulin, simultaneously with, prior to, or after administration of one or more compounds described herein, including compositions comprising them.

Provided is a method of reducing plasma cholesterol levels, in a subject in need thereof, comprising administering an effective amount of at least one compound or pharmaceutically acceptable salt thereof described herein, or a composition comprising at least one compound or pharmaceutically acceptable salt thereof described herein.

Provided is a method of reducing plasma triglyceride levels in a subject in need thereof, comprising administering an effective amount at least one compound or pharmaceutically acceptable salt thereof described herein, or a composition comprising at least one compound or pharmaceutically acceptable salt thereof described herein.

Provided is a method of treating, preventing, inhibiting, or ameliorating one or more symptoms of a disease or disorder which is affected by abnormal cholesterol, triglyceride, or bile acid levels, comprising administering to a subject in need thereof an effective amount of at least one compound or pharmaceutically acceptable salt thereof described herein, or at least one pharmaceutical composition comprising at least one compound or pharmaceutically acceptable salt thereof described herein.

Provided is a method of modulating cholesterol metabolism, catabolism, synthesis, absorption, re-absorption, secretion or excretion in a mammal, comprising administering to a subject in need thereof an effective amount of at least one compound or pharmaceutically acceptable salt thereof described herein, or a composition comprising at least one compound or pharmaceutically acceptable salt thereof described herein.

Provided is a method of treating at least one malignancy in a patient, the method comprising administering to the patient a therapeutically effective amount of at least one compound or pharmaceutically acceptable salt thereof described herein, or a composition comprising at least one compound or pharmaceutically acceptable salt thereof described herein, wherein the at least one compound or pharmaceutically acceptable salt thereof or composition induces expression of the reversion-inducing-cysteine rich-protein with Kazal motifs (RECK) gene in the patient. In some embodiments, the at least one malignancy is selected from hepatocellular carcinoma, colorectal cancer, and breast cancer. In some embodiments, the at least one malignancy is characterized by elevated expression of the human epidermal growth factor receptor 2 (HER2/neu) gene. In some embodiments, the at least one malignancy is selected from hepatocellular carcinoma, colorectal cancer, breast cancer, gastric cancer, renal cancer, salivary gland cancer, ovarian cancer, uterine body cancer, bladder cancer, and lung cancer. In some embodiments the method further comprises coadministering at least one of an agent selected from abarelix, aldeleukin, allopurinol, altretamine, amifostine, anastozole, bevacizumab, capecitabine, carboplatin, cisplatin, docetaxel, doxorubicin, erlotinib, exemestane, 5-fluorouracil, fulvestrant, gemcitabine, goserelin acetate, irinotecan, lapatinib ditosylate, letozole, leucovorin, levamisole, oxaliplatin, paclitaxel, panitumumab, pemetrexed disodium, profimer sodium, tamoxifen, topotecan, and trastuzumab.

Provided is a method of treating nonalcoholic fatty liver disease (NAFLD) in a patient, the method comprising administering to the patient a therapeutically effective amount of at least one compound or pharmaceutically acceptable salt thereof described herein, or a composition comprising at least one compound or pharmaceutically acceptable salt thereof described herein. In some embodiments the nonalcoholic fatty liver disease is characterized by at least one of steatosis, nonalcoholic steatohepatitis (NASH), NAFLD induced hepatitis, NAFLD induced fibrosis, and NAFLD induced cirrhosis. In some embodiments, the at least compound or pharmaceutically acceptable salt thereof or composition reduces at least one feature of nonalcoholic fatty liver disease selected from neutral lipid deposition, intracellular lipid droplet formation, Kupffer cell activation, inflammatory cell infiltration, inflammatory cholangitis, portal inflammation, fibrosis, and oxidative stress in the liver.

Provided is a method of treating a patient with existing cholesterol gallstone disease, wherein the existing cholesterol gallstone disease is characterized by at least one of neutral lipid deposition, intracellular lipid droplet formation, Kupffer cell activation, inflammatory cell infiltration, inflammatory cholangitis, portal inflammation, fibrosis, oxidative stress in the liver, and an elevated level of at least one of VCAM-1, ICAM-1, TNFα, MCP-1, KC, TIMP-1, MMP-9, MMP-14, CYP2E1, ALT, AST, and CK-18, the method comprising administering to the patient a therapeutically effective amount of at least one compound or pharmaceutically acceptable salt thereof described herein, or a composition comprising at least one compound or pharmaceutically acceptable salt thereof described herein. In some embodiments the patient is characterized by at least one feature selected from is highly symptomatic, is awaiting a cholecystectomy, and is not a suitable candidate for surgical intervention.

Also provided is a method of treating at least one disease state characterized by elevated expression of the Lectin-like Oxidized Low-density Lipoprotein Receptor 1 (LOX-1) in a patient, the method comprising administering to the patient a therapeutically effective amount of at least one compound or pharmaceutically acceptable salt thereof described herein, or a composition comprising at least one compound or pharmaceutically acceptable salt thereof described herein, wherein the at least one compound or pharmaceutically acceptable salt thereof or composition reduces expression of LOX-1 in the patient. In some embodiments the disease state is further characterized by at least one of endothelial dysfunction and vascular inflammation. In some embodiments the at least one disease state is selected from heart failure, myocardial injury, atherosclerosis, diabetic nephropathy, hypertension, sepsis, osteoarthritis and rheumatoid arthritis. In some embodiments the heart failure comprises at least one of left sided heart failure, right sided heart failure, systolic heart failure, and diastolic heart failure. In some embodiments the myocardial injury comprises at least one of unstable angina and myocardial infarction. In some embodiments LOX-1 expression is reduced in at least one tissue of the patient selected from heart, liver, and kidney. In some embodiments LOX-1 expression is reduced in at least one cell type of the patient selected from endothelial cells, macrophages, smooth muscle cells, dendritic cells, cardiac myocytes, and platelets. In some embodiments the level of serum soluble LOX-1 protein in the patient is reduced.

Provided is a method of treating at least one condition that can be treated by elevating the vitamin D receptor (VDR) activity level in a patient by administering to the patient a therapeutically effective amount of at least one farnesoid X receptor (FXR) agonist. In some embodiments, the at least one FXR agonist elevates the level of Cytochrome P450, family 27, subfamily B, polypeptide 1 (CYP27B1), to thereby elevate the level of VDR activity in the patient. In some embodiments, the at least one condition is a disease characterized by deficient VDR activity levels in the patient. In some embodiments, the level of CYP27B1 is elevated in at least one cell type of the patient selected from kidney cells and bone cells. In some embodiments, the level of CYP27B1 is elevated in at least one bone cell type of the patient selected from osteoblasts and osteoclasts. In some embodiments, the at least one FXR agonist elevates the level of CYP27B1, to thereby elevate the level of 1α,25-dihydroxyvitamin D3 in at least one of serum of the patient and a cell type of the patient selected from kidney cells and bone cells. In some embodiments, the level of 1α,25-dihydroxyvitamin D3 is elevated in at least one bone cell type of the patient selected from osteoblasts and osteoclasts. In some embodiments, the VDR activity level is elevated in at least one cell type of the patient selected from kidney cells, cardiomyocytes, bone cells, immune cells, mesangial cells, and smooth muscle cells. In some embodiments, the VDR activity level is elevated in at least one bone cell type of the patient selected from osteoblasts and osteoclasts. In some embodiments, the VDR activity level is elevated in at least one immune cell type of the patient selected from dendritic cells, T lymphocytes, B lymphocytes, and monocytes. In some embodiments, administration of the at least one FXR agonist does not cause at least one of hypercalcemia and hypercalcinuria in the patient. In some embodiments, the at least one condition is selected from obesity, glucose intolerance, diabetes, and metabolic syndrome. In some embodiments, the at least one condition is chronic kidney disease. In some embodiments, the chronic kidney disease is characterized by at least one of diabetic nephropathy and renal failure. In some embodiments, treatment of the chronic kidney disease comprises treatment of at least one secondary disorder in the patient selected from parahyperthyroidism and cardiovascular disease. In some embodiments, the cardiovascular disease is characterized by at least one of coronary heart disease, cerebrovascular disease, peripheral vascular disease, congestive heart failure, myocardial infarction, left ventricular hypertrophy, hypertension, and atherosclerosis. In some embodiments, the at least one FXR agonist reduces the level of at least one of a matrix metalloprotease (MMP), an extracellular matrix protein, renin angiotensin system (RAS) pathway, parathyroid hormone, serum creatinine, serum albumin, proteinuria, lipid metabolism, renal lipid deposition, mesangial expansion, glomerulosclerosis, and kidney inflammation in the patient. In some embodiments, the at least one MMP is selected from MMP-9 and MMP-14. In some embodiments, the at least one extracellular matrix protein is selected from collagen IV and fibronectin. In some embodiments, the level of the RAS pathway is characterized by the level of renin in the patient. In some embodiments, the proteinuria is characterized by albuminuria in the patient. In some embodiments, the at least one condition is cardiovascular disease. In some embodiments, the cardiovascular disease is characterized by at least one of coronary heart disease, cerebrovascular disease, peripheral vascular disease, congestive heart failure, myocardial infarction, left ventricular hypertrophy, hypertension, and atherosclerosis. In some embodiments, the at least one FXR agonist reduces the level of at least one of a MMP, parathyroid hormone, blood pressure, and RAS pathway in the patient. In some embodiments, the at least one MMP is selected from MMP-9 and MMP-14. In some embodiments, the level of the RAS pathway is characterized by the level of renin in the patient. In some embodiments, the at least one condition is a bone disease. In some embodiments, the at least one bone disease is characterized by at least one of osteoporosis, osteomalacia, and rickets. In some embodiments, the at least one FXR agonist reduces the level of at least one of parathyroid hormone and bone resorption in the patient. In some embodiments, the at least one FXR agonist elevates the level of bone formation in the patient.

Provided is a method for modulating farnesoid X receptor activity comprising contacting a cell with at least one compound or pharmaceutically acceptable salt thereof described herein or a composition comprising at least one compound or pharmaceutically acceptable salt thereof described herein.

Also provided are pharmaceutical compositions for treating or modulating vascular permeability comprising at least one compound or pharmaceutically acceptable salt thereof described herein, and a pharmaceutically acceptable carrier therefore. Such compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

Liquid carriers may be used in preparing solutions, suspensions, emulsions, syrups and elixirs including intravenous solutions. The active ingredient can be dissolved or suspended in a pharmaceutically acceptable liquid carrier such as water, organic solvent, or a mixture of both. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, osmo-regulators, antioxidants and antifoaming agents.

Suitable examples of liquid carriers for oral, intravenous and parenteral administration include water (particularly containing additives as above e.g., cellulose derivatives, such as sodium carboxymethyl cellulose solution), saline, dextrose solutions, dextrose-saline and dextrose-water solutions, alcohols (including monohydric alcohols and polyhydric alcohols e.g., glycols) and their derivatives. Liquid carriers are used in sterile form for parenteral and intravenous administration. PH of liquid formulations may be adjusted in some cases by the addition of HCl, sodium hydroxide, and phosphoric acid. In certain embodiments, compositions are liquid pharmaceutical compositions which are sterile solutions or suspensions in an iso-osmotic, physiologically compatible buffered system.

Liquid pharmaceutical compositions can be administered by, for example, intramuscular, intraperitoneal, intravenous, or subcutaneous injection. Pharmaceutical compositions may be administered, in certain embodiments, to a patient by intraperitoneal or intravenous injection. In certain embodiments, the composition is administered intravenously such as by intravenous bolus injection, intravenous i.v. drip, repeated slow bolus administration or infusion.

Oral administration may be either liquid or solid composition form. The compounds described herein may also be administered orally or parentally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or an encapsulating material. In powders, the carrier is a finely divided solid, which is in admixture with the finely divided active ingredient. In tablets, the active ingredient is mixed with a carrier having the necessary compression properties in suitable proportions and compacted in the shape and size desired. The powders and tablets may contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

In certain embodiments, the pharmaceutical composition is in unit dosage form, e.g., as tablets, capsules, powders, solutions, suspensions, emulsions, granules, suppositories, ampule, or bolus. In such form, the composition is sub-divided in unit dose containing appropriate quantities of the active ingredient; the unit dosage forms can be packaged compositions, for example packeted powders, lyophilized powder or cake in ampoules or vials, or vials, ampoules, prefilled syringes or sachets containing liquids. The unit dosage form can be, for example, capsule or tablet itself, or it can be the appropriate number of any such compositions in package form.

The dose provided to a patient will vary depending upon what is being administered, the purpose of the administration, such as prophylaxis or therapy, and the state of the patient, the manner of administration, and the like. Generally, a single dose (or dosage form) will contain from about 1 mg/kg to about 30 mg/kg, such as from about 1 mg/kg to about 10 mg/kg of compound described herein. It is expected that some patients will receive multiple doses. The dosage to be used in the treatment of a specific case must be subjectively determined by the attending physician. The variables involved include the specific condition and the size, age and response pattern of the patient.

It is understood that the effective dosage of the active compounds described herein may vary depending upon the particular compound utilized, the mode of administration, the condition being treated and severity thereof, as well as the various physical factors related to the individual being treated. It is projected that compounds described herein will be administered at an oral daily dosage of from about 0.05 mg to about 30 mg per kilogram of body weight, in some embodiments, administered in divided doses two to six times per day, or in a sustained release form, and may be adjusted to provide the optimal therapeutic result.

This invention will be more fully described in conjunction with the following specific examples which are not to be construed as limiting the scope of this invention.

### EXPERIMENTAL EXAMPLES

### Example 1

### Isopropyl-1,1-dimethyl-3-[4-(morpholin-4-ylmethyl)benzoyl]-1,2,3,6-tetrahydroazepino[4,5-b]indole-5-carboxylate

Step 1: A solution of Isopropyl-1,1-dimethyl-1,2,3,6-tetrahydroazepino[4,5-b]indole-5-carboxylate (0.30 g, 1.0 mmol) in dry acetonitrile (15 ml) under nitrogen was cooled to 0° C. To this were added triethylamine (0.17 ml, 1.2 mmol) and 4-(chloromethyl)benzoyl chloride (0.28 g, 1.5 mmol). The reaction mixture was stirred for 1 h at room temperature until the reaction was complete. The mixture was filtered and the filtrate was concentrated under reduced pressure. The resulting crude product was purified via Isco (RediSep Flash Column 12 g, silica, gradient from 5% ethyl acetate/hexane to 30% ethyl acetate/hexane) to give isopropyl 3-[4-(chloromethyl)benzoyl]-1,1-dimethyl-1,2,3,6-tetrahydroazepino[4,5-b]indole-5-carboxylate as a light yellow solid. MS (ES) m/z 451.1 ([M+H]⁺).

Step 2: A solution of isopropyl 3-[4-(chloromethyl)benzoyl]-1,1-dimethyl-1,2,3,6-tetrahydro azepino[4,5-b]indole-5-carboxylate (0.24 g, 0.53 mol) and triethylamine (0.09 ml, 0.64 mmol) in dry acetonitrile (10 mL) was stirred at 0° C. To this was added morpholine (0.07 mL, 0.80 mmol) and the reaction mixture was stirred at room temperature for 5 h until the reaction was complete. The reaction mixture was then washed with a saturated aqueous solution of sodium bicarbonate, dried over anhydrous sodium sulfate, filtered, and concentrated under reduced pressure. The resulting crude product was purified via Isco (RediSep Flash Column 12 g, silica, gradient from 10% to 80% of 2.5% methanol-dichloromethane/dichloromethane) to give a yellow solid as an expected product. The product was recrystallized from ethyl acetate to afford the titled compound, isopropyl 1,1-dimethyl-3-[4-(morpholin-4-ylmethyl)benzoyl]-1,2,3,6-tetrahydroazepino[4,5-b]indole-5-carboxylate as a light yellow solid. MS (ES) m/z 502.2; HRMS: calcd for C₃₀H₃₅N₃O₄+H⁺, 502.2700; found (ESI, [M+H]⁺), 502.2704.

### Example 2

### Isopropyl 8-fluoro-1,1-dimethyl-3-[4-(morpholin-4-ylmethyl)benzoyl]-1,2,3,6-tetrahydroazepino[4,5-b]indole-5-carboxylate

Step 1: In a round bottom flask with stir bar were combined acetic acid (40 mL), formaldehyde (9.3 mL, 114 mmol, 37% soln in water), and dimethylamine (20.0 mL, 178 mmol, 40% soln in water). The solution was cooled to 0° C. in an ice water bath and 6-fluoroindole (15 g, 111 mmol) was added in portions over one hour. After complete addition the ice bath was removed and the reaction warmed gradually to room temperature overnight, after which time the reaction mixture was poured into ice water. The pH was adjusted to 12 with an aqueous solution of sodium hydroxide. The resulting mixture was extracted with diethyl ether (2×). The combined extracts were dried over sodium sulfate and concentrated to dryness. Further drying under vacuum provided 1-(6-fluoro-1H-indol-3-yl)-N,N-dimethylmethanamine as a white solid, which was used without further purification in the next step. MS (ES) m/z 191.0 ([M−H]⁻).

Step 2: In a round bottom flask with stir bar under nitrogen was placed 1-(6-fluoro-1H-indol-3-yl)-N,N-dimethylmethanamine (21.03 g, 109.4 mmol). THF (200 mL) was added with stirring until all solid was in solution. In one portion iodomethane (17 mL, 273.5 mmol) was added. Almost immediately a yellow precipitate formed. Stirring was continued overnight, after which time the solution was concentrated to dryness to provide 1-(6-fluoro-1H-indol-3-yl)-N,N,N-trimethylmethanaminium iodide, which was used in the next step without further purification.

Step 3: In a round bottom flask with stir bar under nitrogen was placed 1-(6-fluoro-1H-indol-3-yl)-N,N,N-trimethylmethanaminium iodide (26 g, 77.8 mmol), and ethanol (200 mL). With stirring, water (50 mL) was added, followed by sodium cyanide (11.4 g, 233.4 mmol). The reaction was heated at 65° C. in an oil bath for 4 h, after which time LC/MS showed no starting material remaining. The reaction was cooled to room temperature and concentrated to a small volume under reduced pressure. The crude mixture was diluted with ethyl acetate (300 mL) and washed with water followed by brine. The ethyl acetate was dried (MgSO₄), filtered, and concentrated to dryness to provide the desired product, (6-fluoro-1H-indol-3-yl)acetonitrile, as an orange-brown oil. MS (ES) m/z 174.8 ([M+H]⁺).

Step 4: In a round bottom flask (6-fluoro-1H-indol-3-yl)acetonitrile (9.0 g, 51.7 mmol) was dissolved in THF (129 mL). With stirring, 4-dimethylaminopyridine (379 mg, 3.1 mmol) and the triethylamine (8.3 mL, 59.5 mmol) were added. To the stirring solution di-tert-butyl dicarbonate (1M in THF, 59.5 mL, 59.5 mmol) was added and stirred overnight. The reaction was then concentrated to a small volume under reduced pressure and diluted with ethyl acetate (300 mL) and washed with water, followed by a 1N aqueous solution of HCl, then brine. The organic layer was dried (MgSO₄), filtered, and concentrated to dryness to provide the desired product, tert-butyl 3-(cyanomethyl)-6-fluoro-1H-indole-1-carboxylate, as a white solid. MS (ES) m/z 274.9 ([M+H]⁺).

Step 5: In a round bottom flask with stir bar DMF (60 mL) was cooled to 0° C. in an ice bath and sodium hydride (2.9 g, 71.0 mmol) was added in portions. In a second round bottom flask containing DMF (60 mL), tert-butyl 3-(cyanomethyl)-6-fluoro-1H-indole-1-carboxylate (7.95 g, 28.4 mmol), and iodomethane (4.42 mL, 71.0 mmol) were combined. This resulting solution was added to the sodium hydride/DMF slurry via cannulae over 30 mins. After addition was completed the reaction was warmed to room temperature. After one hour at room temperature LC/MS showed complete conversion. The reaction was carefully quenched with a saturated aqueous solution of ammonium chloride (*FOAMING*). The aqueous mixture was diluted with ethyl acetate (500 mL) and separated. The ethyl acetate layer was washed with water and brine, then dried (MgSO₄), filtered, and concentrated to give tert-butyl 3-(1-cyano-1-methylethyl)-6-fluoro-1H-indole-1-carboxylate as white needle-shaped crystals. MS (ES) m/z 302.9 ([M+H]⁺).

Step 6: In a round bottom flask tent-butyl 3-(1-cyano-1-methylethyl)-6-fluoro-1H-indole-1-carboxylate (9.0 g) was diluted with dichloromethane (50 mL) and stirred. Trifluoroacetic acid (25 mL) was added with stirring which was continued for six hours. The reaction was concentrated to a small volume under reduced pressure. The crude mixture was diluted with ethyl acetate (300 mL) and washed with a saturated aqueous solution of sodium carbonate (2×) followed by brine (1×). The organic extract was dried (MgSO₄), filtered, and concentrated to dryness providing 2-(6-fluoro-1H-indol-3-yl)-2-methylpropanenitrile, as a tan solid. MS (ES) m/z 203.1 ([M+H]⁺); HRMS: calcd for C₁₂H₁₁FN₂+H+, 203.0979; found (ESI, [M+H]⁺), 203.0987.

Step 7: In a round bottom flask 2-(6-fluoro-1H-indol-3-yl)-2-methylpropanenitrile (5.86 g, 29.0 mmol) was dissolved in THF (200 mL) with stirring and cooled to 0° C. in an ice water bath. Lithium aluminum hydride (2M in THF, 29 mL, 58.0 mmol) was added drop-wise over 20 min and stirred at 0° C. for an additional 30 min. The reaction was warmed to room temperature and a reflux condenser was attached for heating at reflux overnight. After this time the reaction was cooled to room temperature and carefully quenched with sodium sulfate decahydrate (foaming was noted). The precipitate was removed by filtration on filter paper through a ceramic buchner funnel. The filter paper was washed thoroughly with THF. The filtrate was concentrated to dryness under reduced pressure to provide 2-(6-fluoro-1H-indol-3-yl)-2-methylpropan-1-amine, as a gummy brown-orange solid. MS (ES) m/z 207.1 ([M+H]⁺); HRMS: calcd for C₁₂H₁₅FN₂+H+, 207.1292; found (ESI, [M+H]⁺), 207.1296.

Conversion to hydrochloride salt: 2-(6-Fluoro-1H-indol-3-yl)-2-methylpropan-1-amine was dissolved in diethyl ether. In a separate flask HCl gas was bubbled through diethyl ether for 10 min. The acidic solution was added to the solution of 2-(6-fluoro-1H-indol-3-yl)-2-methylpropan-1-amine. The resulting precipitate was isolated by carefully removing the ether. The precipitate was washed with ether and dried under vacuum to provide 2-(6-fluoro-1H-indol-3-yl)-2-methylpropan-1-amine hydrochloride, as light yellow solid, which was used in the next reaction without additional purification.

Step 8: In a round bottom flask with stir bar 2-(6-fluoro-1H-indol-3-yl)-2-methylpropan-1-amine hydrochloride (16.5 g, 68 mmol) was dissolved in isopropanol (160 mL), and acetonitrile (160 mL). Isopropyl bromopyruvate (17.8 g, 85 mmol) was added and the reaction was heated at 80° C. overnight. Pyridine (15.4 mL, 190.4 mmol) and 4-dimethylaminopyridine (0.83 g, 6.8 mmol) and stirred overnight at 80° C. The reaction was concentrated to a small volume under reduced pressure and diluted with dichloromethane (250 mL). The organic layer was washed with a saturated aqueous solution of sodium bicarbonate, followed by water, then brine. The organic extract was dried (MgSO₄), filtered, and concentrated to dryness. Purification of the crude material by flash column using 20 to 80% dichloromethane in hexane as eluant provided isopropyl 8-fluoro-1,1-dimethyl-1,2,3,6-tetrahydroazepino[4,5-b]indole-5-carboxylate as an orange solid. MS (ES) m/z 317.0 ([M+H]⁺); HRMS: calcd for C₁₈H₂₁FN₂O₂+H+, 317.1660; found (ESI, [M+H]⁺), 317.1666.

Step 9: In an analogous manner to Example 1, step 1, isopropyl 3-[4-(chloromethyl)benzoyl]-8-fluoro-1,1-dimethyl-1,2,3,6-tetrahydroazepino[4,5-b]indole-5-carboxylate was prepared from isopropyl-8-fluoro-1,1-dimethyl-1,2,3,6-tetrahydroazepino[4,5-b]indole-5-carboxylate and 4-(chloromethyl)benzoyl chloride as a yellow solid. MS (ES) m/z 469.0 ([M+H]⁺).

Step 10: In an analogous manner to Example 1, step 2, the titled compound, isopropyl 8-fluoro-1,1-dimethyl-3-[4-(morpholin-4-ylmethyl)benzoyl]-1,2,3,6-tetrahydroazepino[4,5-b]indole-5-carboxylate, was prepared from isopropyl 3-[4-(chloromethyl)benzoyl]-8-fluoro-1,1-dimethyl-1,2,3,6-tetrahydroazepino[4,5-b]indole-5-carboxylate and morpholine as a yellow solid. MS (ES) m/z 520.3 ([M+H]⁺); HRMS: calcd for C₃₀H₃₄FN₃O₄+H⁺, 520.2606; found (ESI, [M+H]⁺), 520.2610.

### Example 3

### Isopropyl 1,1-dimethyl-3-[4-(2-morpholin-4-ylethyl)benzoyl]-1,2,3,6-tetrahydroazepino[4,5-b]indole-5-carboxylate hydrochloride

Step 1: Isopropyl-1,1-dimethyl-1,2,3,6-tetrahydroazepino[4,5-b]indole-5-carboxylate (0.45 g, 1.5 mmol) was dissolved in dry dichloromethane (20 mL) under nitrogen and treated with triethylamine (0.21 mL, 1.8 mmol). To this were added EDC (0.52 g, 1.8 mmol) and 4-(2-chloroethyl)benzoic acid (0.33 g, 1.8 mmol) and the reaction mixture was stirred at room temperature for 3 days. The mixture was then washed with water, dried over anhydrous sodium sulfate, filtered, and concentrated under reduced pressure. The resulting residue was purified via Isco (RediSep Flash Column 12 g, silica, gradient from 5% ethyl acetate/hexane to 20% ethyl acetate/hexane) to give a yellow solid as an expected product. The product was recrystallized from n-hexane by adding a minimum amount of ethyl acetate to give isopropyl 3-[4-(2-chloroethyl)benzoyl]-1,1-dimethyl-1,2,3,6-tetrahydroazepino[4,5-b]indole-5-carboxylate as a light yellow solid. MS (ES) m/z 465.1 ([M+H]⁺); HRMS: calcd for C₂₇H₂₉ClN₂O₃+H⁺, 465.1939; found (ESI, [M+H]⁺), 465.1939.

Step 2: To a solution of isopropyl 3-[4-(2-chloroethyl)benzoyl]-1,1-dimethyl-1,2,3,6-tetrahydroazepino[4,5-b]indole-5-carboxylate (0.102 g, 0.22 mmol) and morpholine (1.0 ml, 11 mmol) were added potassium iodide (0.037 g, 0.22 mmol) and triethylamine (0.035 ml, 0.26 mmol). The reaction mixture was stirred at 60° C. for 24 h and cooled to room temperature. The cooled reaction mixture was partitioned between a saturated aqueous solution of sodium bicarbonate and dichloromethane. The separated dichloromethane layer was washed with water, dried over anhydrous sodium sulfate, filtered, and concentrated under reduced pressure. The resulting crude product was purified via Isco (RediSep Flash Column 12 g, silica, gradient from 5% to 50% of 2.5% methanol-dichloromethane/dichloromethane) to give a free base of the expected product as a yellow solid. The free base of the product was dissolved in a minimum amount of ethyl acetate and treated with a 4 N solution of hydrochloride in dioxane until the pH of the solution was 3. The product was then crystallized from ethyl acetate to afford the titled compound, isopropyl 1,1-dimethyl-3-[4-(2-morpholin-4-ylethyl)benzoyl]-1,2,3,6-tetrahydroazepino[4,5-b]indole-5-carboxylate hydrochloride as a light yellow solid. MS (ES) m/z 516.3 [M+H]⁺; HRMS: calcd for C₃₁H₃₇N₃O₄+H⁺, 516.2857; found (ESI, [M+H]⁺), 516.2861.

### Example 4

### Isopropyl 8-fluoro-1,1-dimethyl-3-[4-(2-morpholin-4-ylethyl)benzoyl]-1,2,3,6-tetrahydroazepino[4,5-b]indole-5-carboxylate

In an analogous manner to Example 3, step 1, isopropyl 3-[4-(chloroethyl)benzoyl]-8-fluoro-1,1-dimethyl-1,2,3,6-tetrahydroazepino[4,5-b]indole-5-carboxylate was prepared from isopropyl-8-fluoro-1,1-dimethyl-1,2,3,6-tetrahydroazepino[4,5-b]indole-5-carboxylate (from Example 2) and 4-(2-chloroethyl)benzoic acid as a yellow solid. MS (ES) m/z 483 ([M+H]⁺).

In an analogous manner to Example 3, step 2, isopropyl 8-fluoro-1,1-dimethyl-3-[4-(2-morpholin-4-ylethyl)benzoyl]-1,2,3,6-tetrahydroazepino[4,5-b]indole-5-carboxylate was prepared from Isopropyl 3-[4-(chloroethyl)benzoyl]-8-fluoro-1,1-dimethyl-1,2,3,6-tetrahydroazepino[4,5-b]indole-5-carboxylate and morpholine as a light yellow solid. MS (ES) m/z 534.3 ([M+H]⁺).

### Example 5

### Isopropyl 3-(3-{[(2R,6R)-2,6-dimethylmorpholin-4-yl]methyl}benzoyl)-1,1-dimethyl-1,2,3,6-tetrahydroazepino[4,5-b]indole-5-carboxylate

In an analogous manner, isopropyl 3-(3-{[(2R,6R)-2,6-dimethylmorpholin-4-yl]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 2,6-dimethylmorpholine. The crude product was then purified via Isco (RediSep Flash Column 4 g, silica, gradient from 0% ethyl acetate/hexane to 100% ethyl acetate/hexane) to give the titled compound as a pale yellow solid. MS (ES) m/z 530.1 ([M+H]⁺); HRMS: calcd for C₃₂H₃₉N₃O₄+H+, 530.3013; found (ESI, [M+H]⁺), 530.3013.

### Example 6

### Isopropyl 3-(3-{[(2R,6S)-2,6-dimethylmorpholin-4-yl]methyl}benzoyl)-1,1-dimethyl-1,2,3,6-tetrahydroazepino[4,5-b]indole-5-carboxylate

In an analogous manner, isopropyl 3-(3-{[(2R,6S)-2,6-dimethylmorpholin-4-yl]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 2,6-dimethylmorpholine. The crude product was then purified via Isco (RediSep Flash Column 4 g, silica, gradient from 0% ethyl acetate/hexane to 100% ethyl acetate/hexane) to give the titled compound as a pale yellow solid. MS (ES) m/z 530.1 ([M+H]⁺); HRMS: calcd for C₃₂H₃₉N₃O₄+H+, 530.3013; found (ESI, [M+H]⁺), 530.3014.

### Example 7

### Isopropyl 3-(3-{[(3R,5S)-3,5-dimethylpiperazin-1-yl]methyl}benzoyl)-1,1-dimethyl-1,2,3,6-tetrahydroazepino[4,5-b]indole-5-carboxylate

In an analogous manner, isopropyl 3-(3-{[(3R,5S)-3,5-dimethylpiperazin-1-yl]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and cis-2,6-dimethyl piperidine. The crude product was purified via Isco (RediSep Flash Column 4 g, silica, gradient from 0% to 100% of 5.0% methanol-dichloromethane/dichloromethane) to give the titled compound as a yellow solid. MS (ES) m/z 529.1 ([M+H]⁺); HRMS: calcd for C₃₂H₄₀N₄O₃+H+, 529.3173; found (ESI, [M+H]⁺), 529.3175.

### Example 8

### Isopropyl 3-{3-[(4-carbamoylpiperidin-1-yl)methyl]benzoyl}-1,1-dimethyl-1,2,3,6-tetrahydroazepino[4,5-b]indole-5-carboxylate

In an analogous manner, isopropyl 3-{3-[(4-carbamoylpiperidin-1-yl)methyl]benzoyl}-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and piperidine-4-carboxamide as a pale yellow solid. MS (ES) m/z 543.2 ([M+H]⁺); HRMS: calcd for C₃₂H₃₈N₄O₄+H+, 543.2966; found (ESI, [M+H]⁺), 543.2966.

### Example 9

### Isopropyl 1,1-dimethyl-3-{3-[(4-morpholin-4-ylpiperidin-1-yl)methyl]benzoyl}-1,2,3,6-tetrahydroazepino[4,5-b]indole-5-carboxylate

In an analogous manner, isopropyl 1,1-dimethyl-3-{3-[(4-morpholin-4-ylpiperidin-1-yl)methyl]benzoyl}-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 4-morpholinopiperidine as a yellow solid. MS (ES) m/z 585.3 ([M+H]⁺); HRMS: calcd for C₃₅H₄₄N₄O₄+H+, 585.3435; found (ESI, [M+H]⁺), 585.3434.

### Example 10

### Isopropyl 1,1-dimethyl-3-[3-(1,3-thiazolidin-3-ylmethyl)benzoyl]-1,2,3,6-tetrahydroazepino[4,5-b]indole-5-carboxylate

In an analogous manner, isopropyl 1,1-dimethyl-3-[3-(1,3-thiazolidin-3-ylmethyl)benzoyl]-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and thiazolidine as a pale yellow solid. The product was recrystallized from ethyl acetate to give a free base of the desired product as a pale yellow solid. MS (ES) m/z 504.2 ([M+H]⁺); HRMS: calcd for C₂₉H₃₃N₃O₃S+H+, 504.2315; found (ESI, [M+H]⁺), 504.2315.

### Example 11

### Isopropyl 3-[3-(1,4′-bipiperidin-1′-ylmethyl)benzoyl]-1,1-dimethyl-1,2,3,6-tetrahydroazepino[4,5-b]indole-5-carboxylate

In an analogous manner, isopropyl 3-[3-(1,4′-bipiperidin-1-ylmethyl)benzoyl]-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 4-piperidinopiperidine as a pale yellow solid. MS (ES) m/z 583.3 ([M+H]⁺); HRMS: calcd for C₃₆H₄₆N₄O₃+H+, 583.3643; found (ESI, [M+H]⁺), 583.3642.

### Example 12

### Isopropyl 1,1-dimethyl-3-{3-[(4-pyrrolidin-1-ylpiperidin-1-yl)methyl]benzoyl}-1,2,3,6-tetrahydroazepino[4,5-b]indole-5-carboxylate

In an analogous manner, isopropyl 1,1-dimethyl-3-{3-[(4-pyrrolidin-1-ylpiperidin-1-yl)methyl]benzoyl}-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 4-(1-pyrrolidinyl)piperidine as a pale yellow solid. MS (ES) m/z 569.3 ([M+H]⁺); HRMS: calcd for C₃₅H₄₄N₄O₃+H+, 569.3486; found (ESI, [M+H]⁺), 569.3487.

### Example 13

### Isopropyl 3-(3-{[(3S,5S)-3,5-dimethylpiperidin-1-yl]methyl}benzoyl)-1,1-dimethyl-1,2,3,6-tetrahydroazepino[4,5-b]indole-5-carboxylate

In an analogous manner to Example 5, isopropyl 3-(3-{[(3S,5S)-3,5-dimethylpiperidin-1-yl]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 3,5-dimethylpiperidine as a yellow solid. MS (ES) m/z 528.1 ([M+H]⁺); HRMS: calcd for C₃₃H₄₁N₃O₃+H+, 528.3221; found (ESI, [M+H]⁺), 528.3232.

### Example 14

### Isopropyl 3-(3-{[(R3R,5S)-3,5-dimethylpiperidin-1-yl]methyl}benzoyl)-1,1-dimethyl-1,2,3,6-tetrahydroazepino[4,5-b]indole-5-carboxylate

In an analogous manner to Example 6, isopropyl 3-(3-{[(3R,5S)-3,5-dimethylpiperidin-1-yl]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 3,5-dimethylpiperidine as a yellow solid. MS (ES) m/z 528.1 ([M+H]⁺); HRMS: calcd for C₃₃H₄₁N₃O₃+H+, 528.3221; found (ESI, [M+H]⁺), 528.3233.

### Example 15

### Isopropyl 1,1-dimethyl-3-[3-(thiomorpholin-4-ylmethyl)benzoyl]-1,2,3,6-tetrahydroazepino[4,5-b]indole-5-carboxylate

In an analogous manner, isopropyl 1,1-dimethyl-3-[3-(thiomorpholin-4-ylmethyl)benzoyl]-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and thiomorpholine as a yellow solid. MS (ES) m/z 518.0 ([M+H]⁺); HRMS: calcd for C₃₀H₃₅N₃O₃S+H+, 518.2472; found (ESI, [M+H]⁺), 518.2476.

### Example 16

### Isopropyl 3-(3-{[(2S,5S)-2,5-dimethylpyrrolidin-1-yl]methyl}benzoyl)-1,1-dimethyl-1,2,3,6-tetrahydroazepino[4,5-b]indole-5-carboxylate

In an analogous manner to Example 5, isopropyl 3-(3-{[(2S,5S)-2,5-dimethylpyrrolidin-1-yl]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and 2,5-dimethylpyrrolidine as a yellow solid. MS (ES) m/z 514.1 ([M+H]⁺); HRMS: calcd for C₃₂H₃₉N₃O₃+H+, 514.3064; found (ESI, [M+H]⁺), 514.3066.

### Example 17

### Isopropyl 3-{3-[(cyclohexylamino)methyl]benzoyl}-1,1-dimethyl-1,2,3,6-tetrahydroazepino[4,5-b]indole-5-carboxylate

In an analogous manner, isopropyl 3-{3-[(cyclohexylamino)methyl]benzoyl}-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and cyclohexanamine as a yellow solid. MS (ES) m/z 514.1 ([M+H]⁺); HRMS: calcd for C₃₂H₃₉N₃O₃+H+, 514.3064; found (ESI, [M+H]⁺), 514.3066.

### Example 18

### Isopropyl 3-(3-{[cyclohexyl(methyl)amino]methyl}benzoyl)-1,1-dimethyl-1,2,3,6-tetrahydroazepino[4,5-b]indole-5-carboxylate

In an analogous manner, isopropyl 3-(3-{[cyclohexyl(methyl)amino]methyl}benzoyl)-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and N-methylcyclohexanamine as a yellow solid. MS (ES) m/z 528.1 ([M+H]⁺); HRMS: calcd for C₃₃H₄₁N₃O₃+H+, 528.3221; found (ESI, [M+H]⁺), 528.3225.

### Example 19

### Isopropyl 3-{3-[(4-hydroxypiperidin-1-yl)methyl]benzoyl}-1,1-dimethyl-1,2,3,6-tetrahydroazepino[4,5-b]indole-5-carboxylate

In an analogous manner, isopropyl 3-{3-[(4-hydroxypiperidin-1-yl)methyl]benzoyl}-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and piperidin-4-ol as a yellow solid. MS (ES) m/z 516.0 ([M+H]⁺); HRMS: calcd for C₃₁H₃₇N₃O₄+H+, 516.2857; found (ESI, [M+H]⁺), 516.2858.

### Example 20

### isopropyl 3-{3-[(3-carbamoylpiperidin-1-yl)methyl]benzoyl}-1,1-dimethyl-1,2,3,6-tetrahydroazepino[4,5-b]indole-5-carboxylate

In an analogous manner, isopropyl 3-{3-[(3-carbamoylpiperidin-1-yl)methyl]benzoyl}-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and piperidine-3-carboxamide as a yellow solid. MS (ES) m/z 543.1 ([M+H]⁺); HRMS: calcd for C₃₂H₃₈N₄O₄+H+, 543.2966; found (ESI, [M+H]⁺), 543.2965.

### Example 21

### Isopropyl 3-{3-[(1,1-dioxidothiomorpholin-4-yl)methyl]benzoyl}-1,1-dimethyl-1,2,3,6-tetrahydroazepino[4,5-b]indole-5-carboxylate

In an analogous manner, isopropyl 3-{3-[(1,1-dioxidothiomorpholin-4-yl)methyl]benzoyl}-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and thiomorpholine 1,1-dioxide as a yellow solid. MS (ES) m/z 549.9 ([M+H]⁺); HRMS: calcd for C₃₀H₃₅N₃O₅S+H+, 550.2370; found (ESI, [M+H]⁺), 550.2367.

### Example 22

### Isopropyl 1,1-dimethyl-3-{3-[(piperidin-4-ylamino)methyl]benzoyl}-1,2,3,6-tetrahydroazepino[4,5-b]indole-5-carboxylate dihydrochloride

In an analogous manner, isopropyl 3-[3-({[1-(tert-butoxycarbonyl)piperidin-4-yl]amino}methyl)benzoyl]-1,1-dimethyl-1,2,3,6-tetrahydroazepino[4,5-b]indole-5-carboxylate was prepared from isopropyl 3-[3-(chloromethyl)benzoyl]-1,1-dimethyl-1,2,3,6-tetrahydro azepino[4,5-b]indole-5-carboxylate and tert-butyl 4-aminopiperidine-1-carboxylate as a pale yellow solid. MS (ES) m/z 615.2 ([M+H]⁺); HRMS: calcd for C₃₆H₄₆N₄O₅+H⁺, 615.3541; found (ESI, [M+H]⁺), 615.3537.

Isopropyl 3-[3-({[1-(tert-butoxycarbonyl)piperidin-4-yl]amino}methyl)benzoyl]-1,1-dimethyl-1,2,3,6-tetrahydroazepino[4,5-b]indole-5-carboxylate (0.05 g, 0.08 mmol) was dissolved in dichloromethane (3 mL). To this was added trifluoroacetic acid (1.8 mL) at 0° C. and stirred at room temperature for 15 h. The reaction mixture was diluted with dichloromethane (5 mL) and carefully neutralized with a saturated aqueous solution of sodium bicarbonate. The mixture was then extracted with a mixed solution of dichloromethane (3 volume) and isopropyl alcohol (1 volume), washed with water, dried over anhydrous sodium sulfate, filtered, and concentrated under reduced pressure. The resulting crude product was dissolved in a minimum amount of dichloromethane and treated with a 2 N solution of hydrochloride in diethyl ether until the solution was pH=3. The product was then crystallized from dichloromethane by adding a minimum amount of diethyl ether to afford the titled compound, isopropyl 1,1-dimethyl-3-{3-[(piperidin-4-ylamino)methyl]benzoyl}-1,2,3,6-tetrahydroazepino[4,5-b]indole-5-carboxylate dihydrochloride, as a yellow solid. MS (ES) m/z 515.0 ([M+H]⁺); HRMS: calcd for C₃₁H₃₈N₄O₃+H+, 515.3017; found (ESI, [M+H]⁺), 515.3018.

### Example 23

### Isopropyl 1,1-dimethyl-3-{4-[2-(4-methylpiperazin-1-yl)ethoxy]benzoyl}-1,2,3,6-tetrahydroazepino[4,5-b]indole-5-carboxylate

Step 1: To a solution of methyl-4-hydroxybenzoate (5 g, 32.9 mmol) in acetone (50 mL) was added benzyl bromide (5.63 g, 32.9 mmol) and potassium carbonate (9.08 g, 65.7 mmol) according to a literature procedure (Bioorg. Med. Chem. 2003, 1381-1387). The reaction was heated at 56° C. for 12 h. The reaction was concentrated under reduced pressure. The crude was partitioned between ethyl acetate (100 mL) and water (100 mL). The ethyl acetate layer was washed with water (2×) and dried of MgSO₄ and concentrated to provide methyl 4-(benzyloxy)benzoate as a white solid. MS (ES) m/z 243.1 ([M+H]⁺).

Step 2: Methyl 4-(benzyloxy)benzoate (6.99 g, 28.9 mmol) was dissolved in methanol (100 mL) and a 30% aqueous solution of potassium hydroxide (20 mL) was added and heated at 64° C. for 6 h. The solvent was evaporated and the crude mixture was treated with ice water (250 mL). The pH was adjusted to 1 with the addition of 6 N HCl. The precipitate was collected on filter paper via vacuum filtration. The white solid was dried on a vacuum pump overnight to provide 4-(benzyloxy)benzoic acid as a white solid. MS (ES) m/z 229.0 ([M+H]⁺).

Step 3: A solution of 4-(benzyloxy)benzoic acid (1.37 g, 6 mmol) and thionyl chloride (12 mL) was heated at 78° C. for 4 h. The reaction was concentrated under reduced pressure. Toluene was added and the resulting solution was dried in vacuo. The toluene evaporation procedure was repeated (2×). The crude 4-(benzyloxy)benzoyl chloride was dried under vacuum for 2 h and used without further purification in the next reaction.

Step 4: A solution of isopropyl-1,1-dimethyl-1,2,3,6-tetrahydroazepino[4,5-b]indole-5-carboxylate (1.2 g, 4 mmol) in dry acetonitrile (20 mL) and triethylamine (837 μL, 6 mmol) was added to a solution of 4-(benzyloxy)benzoyl chloride (6 mmol, from Step 3) in dry acetonitrile (15 mL). The reaction mixture was stirred for 1 hour at room temperature after which distilled water (40 mL) was added. The resulting precipitate was collected by vacuum filtration and recrystallized from ethyl acetate:hexane (5:1, 12 mL) to provide isopropyl 3-(4-(benzyloxy)benzoyl)-1,1-dimethyl-1,2,3,6-tetrahydroazepino[4,5-b]indole-5-carboxylate as a yellow solid. MS (ES) m/z 509 ([M+H]⁺).

Step 5: Isopropyl 3-(4-(benzyloxy)benzoyl)-1,1-dimethyl-1,2,3,6-tetrahydroazepino[4,5-b]indole-5-carboxylate (200 mg, 393 μmol) was dissolved in a mixture of methanol (9 mL) and ethyl acetate (1 mL). Next, 1,4-cyclohexadiene (375 μL, 3.93 mmol) and palladium hydroxide (20% on carbon, 100 mg) were added under nitrogen and the mixture was capped (sealed tube). The reaction was stirred at 64° C. for 1-2 h until judged complete by LC/MS. The reaction was cooled to rt and filtered through Celite. The Celite was rinsed with methanol (3×10 mL) and the combined filtrate was concentrated. The crude mixture was treated with diethylether:hexane (1:1) (3 mL) to provide the crystalline yellow product, isopropyl 3-(4-hydroxybenzoyl)-1,1-dimethyl-1,2,3,6-tetrahydroazepino[4,5-b]indole-5-carboxylate. MS (ES) m/z 418.9 ([M+H]⁺); HRMS: calcd for C₂₅H₂₆N₂O₄+H+, 419.1965; found (ESI, [m+H]⁺), 419.1970.

Step 6: To a solution of isopropyl 3-(4-hydroxybenzoyl)-1,1-dimethyl-1,2,3,6-tetrahydroazepino[4,5-b]indole-5-carboxylate (80 mg, 191 μmol) in THF (2 mL) was added 2-bromoethanol (25 μL, 348 μmol), triphenylphosphine (91.3 mg, 348 μmol), and diethylazodicarboxylate (54.1 μL, 344 μmol). The reaction was stirred under nitrogen at ambient temperature for 18 h. The crude reaction was concentrated and diluted with diethyl ether (10 mL) and washed with water (3×10 mL). The organic layer was dried over MgSO4, filtered, and concentrated under reduced pressure. The resulting residue was purified via Isco (RediSep Flash Column 12 g, silica, gradient from 5% ethyl acetate/hexane to 25% ethyl acetate/hexane) to give the desired yellow solid, isopropyl 3-[4-(2-bromoethoxy)benzoyl]-1,1-dimethyl-1,2,3,6-tetrahydroazepino[4,5-b]indole-5-carboxylate. MS (ES) m/z 524.9, 526.8 ([M+H]⁺).

Step 7: To a solution of isopropyl 3-[4-(2-bromoethoxy)benzoyl]-1,1-dimethyl-1,2,3,6-tetrahydroazepino[4,5-b]indole-5-carboxylate (26.2 mg, 50 μmol) in 1-methyl-2-pyrrolidinone (0.5 mL) was added N-methylpiperazine (30 mg, 300 μmol). The reaction was stirred at ambient temperature for 18 h. The crude reaction was transferred to a Gilson sample tube containing triethylamine (50 μL) and methanol (200 μL). The reaction vessel was rinsed into the sample tube with methanol (400 μL followed by 200 μL). Distilled water (200 μL) was added to the sample tube and the crude was purified by RP-HPLC using a Gilson automated HPLC system and collector: Column; Sunfire prep C18, 5μ, 19×50 mm. Isocratic 10/90 Acetonitrile/Water (10 mL/min, no modifier) for 1.6 min followed by a gradient to 100% acetonitrile (20 mL/min, no modifier) at 9.5 min; then hold for three min at 100% acetonitrile and ramp back to 10/90 acetonitrile/water over 2.0 min. The product was collected in sample tubes and analyzed by LC/MS. Fractions containing the desired purified product were combined and lyophilized to give the titled compound, isopropyl 1,1-dimethyl-3-{4-[2-(4-methylpiperazin-1-yl)ethoxy]benzoyl}-1,2,3,6-tetrahydroazepino[4,5-b]indole-5-carboxylate, as a yellow solid. MS (ES) m/z 545.0 ([M+H]⁺); HRMS: calcd for C₃₂H₄₀N₄O₄+H+, 545.3122; found (ESI, [M+H]⁺), 545.3125.

### Example 24

### Isopropyl 1,1-dimethyl-3-{4-[3-(4-methylpiperazin-1-yl)propoxy]benzoyl}-1,2,3,6-tetrahydroazepino[4,5-b]indole-5-carboxylate

In an analogous manner to Example 23, step 6, isopropyl 3-[4-(3-bromopropoxy)benzoyl]-1,1-dimethyl-1,2,3,6-tetrahydroazepino[4,5-b]indole-5-carboxylate was obtained as a yellow solid using 3-bromopropanol in place of 2-bromoethanol. MS (ES) m/z 538.9, 540.8 ([M+H]⁺).

In an analogous manner to example 23, Step 7, isopropyl 1,1-dimethyl-3-{4-[3-(4-methylpiperazin-1-yl)propoxy]benzoyl}-1,2,3,6-tetrahydroazepino[4,5-b]indole-5-carboxylate was prepared from isopropyl 3-[4-(3-bromopropoxy)benzoyl]-1,1-dimethyl-1,2,3,6-tetrahydroazepino[4,5-b]indole-5-carboxylate as a yellow solid. MS (ES) m/z 559.0 ([M+H]⁺); HRMS: calcd for C₃₃H₄₂N₄O₄+H+, 559.3279; found (ESI, [M+H]⁺), 559.3280.

### Example 25

### Isopropyl 1,1-dimethyl-3-[4-(3-pyrrolidin-1-ylpropoxy)benzoyl]-1,2,3,6-tetrahydroazepino[4,5-b]indole-5-carboxylate

In an analogous manner, isopropyl 1,1-dimethyl-3-[4-(3-pyrrolidin-1-ylpropoxy)benzoyl]-1,2,3,6-tetrahydroazepino[4,5-b]indole-5-carboxylate was prepared from isopropyl 3-[4-(3-bromopropoxy)benzoyl]-1,1-dimethyl-1,2,3,6-tetrahydroazepino[4,5-b]indole-5-carboxylate and pyrrolidine as a yellow solid. MS (ES) m/z 530.0 ([M+H]⁺); HRMS: calcd for C₃₂H₃₉N₃O₄+H+, 530.3013; found (ESI, [M+H]⁺), 530.3016.

### Example 26

### Isopropyl 1,1-dimethyl-3-[3-(2-pyrrolidin-1-ylethoxy)benzoyl]-1,2,3,6-tetrahydroazepino[4,5-b]indole-5-carboxylate

Step 1: In an analogous manner to Example 23, step 1, methyl 3-(benzyloxy)benzoate was prepared from methyl-3-hydroxybenzoate as a white solid. MS (ES) m/z[M+H]⁺=243.1; HRMS: calcd for C₁₅H₁₄O₃+H+, 243.1016; found (ESI, [M+H]⁺), 243.1010.

Step 2: In an analogous manner to Example 23, step 2, 3-(benzyloxy)benzoic acid was prepared from methyl 3-(benzyloxy)benzoate as a white solid. MS (ES) m/z [M−H]⁻=227.0; HRMS: calcd for C₁₄H₁₂O₃+H+, 229.0859; found (ESI, [M+H]⁺), 229.0854.

Step 3: In an analogous manner to Example 23, step 3, 3-(benzyloxy)benzoyl chloride was prepared from 3-(benzyloxy)benzoic acid and used without further purification in the next reaction.

Step 4: In an analogous manner to Example 23, step 4, isopropyl 3-(3-(benzyloxy)benzoyl)-1,1-dimethyl-1,2,3,6-tetrahydroazepino[4,5-b]indole-5-carboxylate was prepared from isopropyl-1,1-dimethyl-1,2,3,6-tetrahydroazepino[4,5-b]indole-5-carboxylate and 3-(benzyloxy)benzoyl chloride as a yellow solid. MS (ES) m/z 508.9 ([M+H]⁺); HRMS: calcd for C₃₂H₃₂N₂O₄+H+, 509.2435; found (ESI, [M+H]⁺), 509.2438.

Step 5: In an analogous manner to Example 23, step 5, isopropyl 3-(3-hydroxybenzoyl)-1,1-dimethyl-1,2,3,6-tetrahydroazepino[4,5-b]indole-5-carboxylate was prepared from isopropyl 3-(3-(benzyloxy)benzoyl)-1,1-dimethyl-1,2,3,6-tetrahydroazepino[4,5-b]indole-5-carboxylate as a yellow solid. MS (ES) m/z 418.9 ([M+H]⁺); HRMS: calcd for C₂₅H₂₆N₂O₄+H+, 419.1965; found (ESI, [M+H]⁺), 419.1965.

Step 6: In an analogous manner to Example 23, step 6, isopropyl 3-[3-(2-bromoethoxy)benzoyl]-1,1-dimethyl-1,2,3,6-tetrahydroazepino[4,5-b]indole-5-carboxylate was prepared from isopropyl 3-(3-hydroxybenzoyl)-1,1-dimethyl-1,2,3,6-tetrahydroazepino[4,5-b]indole-5-carboxylate and 2-bromoethanol as a yellow solid. MS (ES) m/z 524.8, 526.8 ([M+H]⁺); HRMS: calcd for C₂₇H₂₉BrN₂O₄+H+, 525.1384; found (ESI, [M+H]⁺), 525.1380.

In an analogous manner to Example 23, step 7, isopropyl 1,1-dimethyl-3-[3-(2-pyrrolidin-1-ylethoxy)benzoyl]-1,2,3,6-tetrahydroazepino[4,5-b]indole-5-carboxylate was prepared from isopropyl 3-[3-(2-bromoethoxy)benzoyl]-1,1-dimethyl-1,2,3,6-tetrahydroazepino[4,5-b]indole-5-carboxylate and pyrrolidine as a yellow solid. MS (ES) m/z 515.9 ([M+H]⁺); HRMS: calcd for C₃₁H₃₇N₃O₄+H+, 516.2857; found (ESI, [M+H]⁺), 516.2850.

### Example 27

### Isopropyl 1,1-dimethyl-3-{3-[2-(4-methylpiperazin-1-yl)ethoxy]benzoyl}-1,2,3,6-tetrahydroazepino[4,5-b]indole-5-carboxylate

In an analogous manner, isopropyl 1,1-dimethyl-3-{3-[2-(4-methylpiperazin-1-yl)ethoxy]benzoyl}-1,2,3,6-tetrahydroazepino[4,5-b]indole-5-carboxylate was prepared from isopropyl 3-[3-(2-bromoethoxy)benzoyl]-1,1-dimethyl-1,2,3,6-tetrahydroazepino[4,5-b]indole-5-carboxylate and N-methylpiperazine as a yellow solid. MS (ES) m/z 544.9 ([M+H]⁺); HRMS: calcd for C₃₂H₄₀N₄O₄+H+, 545.3122; found (ESI, [M+H]⁺), 545.3116.

### Example 28

### Isopropyl 1,1-dimethyl-3-[3-(3-pyrrolidin-1-ylpropoxy)benzoyl]-1,2,3,6-tetrahydroazepino[4,5-b]indole-5-carboxylate

In an analogous manner to Example 26, step 6, isopropyl 3-[3-(3-bromopropoxy)benzoyl]-1,1-dimethyl-1,2,3,6-tetrahydroazepino[4,5-b]indole-5-carboxylate was obtained as a yellow solid using 3-bromopropanol in place of 2-bromoethanol. MS (ES) m/z 538.8, 540.7 ([M+H]⁺).

In an analogous manner to Example 26, step 7, isopropyl 1,1-dimethyl-3-[3-(3-pyrrolidin-1-ylpropoxy)benzoyl]-1,2,3,6-tetrahydroazepino[4,5-b]indole-5-carboxylate was prepared from isopropyl 3-[3-(3-bromopropoxy)benzoyl]-1,1-dimethyl-1,2,3,6-tetrahydroazepino[4,5-b]indole-5-carboxylate and pyrrolidine as a yellow solid. MS (ES) m/z 529.9 ([M+H]⁺); HRMS: calcd for C₃₂H₃₉N₃O₄+H+, 530.3013; found (ESI, [M+H]⁺), 530.3006.

### Example 29

### Isopropyl 1,1-dimethyl-3-{3-[3-(4-methylpiperazin-1-yl)propoxy]benzoyl}-1,2,3,6-tetrahydroazepino[4,5-b]indole-5-carboxylate

In an analogous manner, isopropyl 1,1-dimethyl-3-{3-[3-(4-methylpiperazin-1-yl)propoxy]benzoyl}-1,2,3,6-tetrahydroazepino[4,5-b]indole-5-carboxylate was prepared from isopropyl 3-[3-(3-bromopropoxy)benzoyl]-1,1-dimethyl-1,2,3,6-tetrahydroazepino[4,5-b]indole-5-carboxylate and N-methylpiperazine as a yellow solid. MS (ES) m/z 558.9 ([M+H]⁺); HRMS: calcd for C₃₃H₄₂N₄O₄+H+, 559.3279; found (ESI, [M+H]⁺), 559.3271.

### Example 30

### Isopropyl 8-fluoro-1,1-dimethyl-3-[4-(3-morpholin-4-ylpropoxy)benzoyl]-1,2,3,6-tetrahydroazepino[4,5-b]indole-5-carboxylate

Step 1: To a solution of benzyl-4-hydroxybenzoate (100 mg, 0.438 mmol) and 3-bromo-1-propanol (70 uL, 0.797 mmol) in THF (2 mL) was added diethyl azodicarboxylate (137 uL, 0.789 mmol). Triphenylphosphine (208 mg, 0.797 mmol) was added to the mixture portionwise at room temperature. After 1 h, the reaction was judged complete by LC/MS. The reaction was concentrated to near dryness using a rotovap, diluted with diethyl ether and washed with water (2×). The organic layer was dried (MgSO₄), filtered, concentrated to a small volume and placed in a freezer. After 2 h, triphenylphosphine oxide precipitated out of solution. The solution was decanted, concentrated, and chromatographed (0%-20% gradient of ethyl acetate/hexane) to yield benzyl 4-(3-bromopropoxy)benzoate as a white solid. MS (ES) m/z 348.9, 350.9 ([M+H]⁺).

Step 2: To a solution of benzyl 4-(3-bromopropoxy)benzoate (10 g, 28.7 mmol) in acetonitrile (100 mL) was added morpholine (15 mL, 172 mmol). The reaction was stirred at room temperature for 18 h, at which time the reaction was judged complete by LC/MS. The reaction was concentrated to near dryness and partitioned between ethyl acetate and water. The aqueous layer was extracted with ethyl acetate (1×) and the combined organic layers were washed with saturated sodium bicarbonate (1×), water (1×), and brine (1×). The organic layer was dried (MgSO₄), filtered, and concentrated to yield benzyl 4-(3-morpholin-4-ylpropoxy)benzoate as a clear oil. MS (ES) m/z 356.3 ([M+H]⁺).

Step 3: To benzyl 4-(3-morpholin-4-ylpropoxy)benzoate (800 mg, 2.25 mmol) in methanol (54 mL) and ethyl acetate (6 mL) was added cyclohexadiene (2.14 mL, 22.6 mmol) and palladium hydroxide on carbon—20% (570 mg). The reaction was flushed with nitrogen and capped with a rubber septum. To relieve any pressure build-up that could occur during the reaction, the septum was pierced with a needle attached to an empty balloon. The sealed system was heated at 64° C. for 1 h, at which time the reaction was judged complete by LC/MS. The reaction was filtered through celite, washed with methanol (3×), and concentrated. After drying for 2 additional hours under vacuum the desired product 4-(3-morpholin-4-ylpropoxy)benzoic acid was obtained as an off-white solid. MS (ES) m/z 265.9 ([M+H]⁺).

Step 4: A solution of 4-(3-morpholin-4-ylpropoxy)benzoic acid (600 mg, 2.25 mmol) and thionyl chloride (4.5 mL) was heated at 78° C. for 2.5 h. The reaction was concentrated on a rotovap. Toluene was added and the resulting solution was concentrated. The toluene evaporation procedure was repeated (2×). The crude was dried on a vacuum pump for 1-2 h to provide 4-(3-morpholinopropoxy)benzoyl chloride, which was used in the next reaction without further purification.

Step 5: To 4-(3-morpholinopropoxy)benzoyl chloride (prepared in step 4, 6.5 g, 22.9 mmol) suspended in acetonitrile (160 mL) was added a solution of isopropyl 8-fluoro-1,1-dimethyl-1,2,3,6-tetrahydroazepino[4,5-b]indole-5-carboxylate (preparation described for Example 2, step 8, 4.84 g, 15.3 mmol) and triethylamine (8.5 mL, 61.2 mmol) in acetonitrile (40 mL). After 10 min the reaction was judged to be 50% complete by LC/MS although a precipitate of 4-(3-morpholinopropoxy)benzoyl chloride was noted in the reaction mixture. Sonication of the reaction for 5 min provided improved solubility and after stirring overnight methanol (2 mL) was added and the reaction was concentrated. The crude was partitioned between ethyl acetate (50 mL) and saturated sodium bicarbonate (50 mL). The organic layer was separated and the aqueous layer was extracted again with ethyl acetate. The organic layers were combined and washed with brine (1×) and water (1×) and dried over MgSO₄. The resulting crude product was purified via two separated Isco runs (RediSep Flash Column 120 g, silica, gradient from 0% to 5% methanol-dichloromethane over 28 min). The titled compound, isopropyl 8-fluoro-1,1-dimethyl-3-[4-(3-morpholin-4-ylpropoxy)benzoyl]-1,2,3,6-tetrahydroazepino[4,5-b]indole-5-carboxylate, was obtained by crystallization of the purified material from acetonitrile to yield yellow crystals. MS (ES) m/z 564.2 ([M+H]⁺); HRMS: calcd for C₃₂H₃₈FN₃O₅+H+, 564.2868; found (ESI, [M+H]⁺), 564.2875.

### Example 31

### Isopropyl 8-fluoro-1,1-dimethyl-3-[4-(2-morpholin-4-ylethoxy)benzoyl]-1,2,3,6-tetrahydroazepino[4,5-b]indole-5-carboxylate

In an analogous manner to Example 30, step 1, benzyl 4-(2-bromoethoxy)benzoate, was prepared using diisopropylazodicarboxylate in place of diethylazodicarboxylate and 2-bromoethanol in place of 3-bromopropanol, as a white solid. MS (ES) m/z 334.9, 336.9 ([M+H]⁺).

In an analogous manner to Example 30, step 2, benzyl 4-(2-morpholin-4-ylethoxy)benzoate was prepared from benzyl 4-(2-bromoethoxy)benzoate and morpholine as a white solid. MS (ES) m/z 341.9 ([M+H]⁺); HRMS: calcd for C₂₀H₂₃NO₄+H+, 342.1700; found (ESI, [M+H]⁺), 342.1705.

In an analogous manner to Example 30, step 3, 4-(2-morpholin-4-ylethoxy)benzoic acid was prepared from benzyl 4-(2-morpholin-4-ylethoxy)benzoate as an off-white solid. MS (ES) m/z 252.1 ([M+H]⁺); HRMS: calcd for C₁₃H₁₇NO₄+H+, 252.1230; found (ESI, [M+H]⁺), 252.1238.

In an analogous manner to Example 30, step 4, 4-(2-morpholin-4-ylethoxy)benzoyl chloride was prepared from 4-(2-morpholin-4-ylethoxy)benzoic acid as an off-white solid. This compound was used in the next reaction without purification.

In an analogous manner to Example 30, step 5, the titled compound, isopropyl 8-fluoro-1,1-dimethyl-3-[4-(2-morpholin-4-ylethoxy)benzoyl]-1,2,3,6-tetrahydroazepino[4,5-b]indole-5-carboxylate, was prepared from isopropyl 8-fluoro-1,1-dimethyl-1,2,3,6-tetrahydroazepino[4,5-b]indole-5-carboxylate and 4-(2-morpholin-4-ylethoxy)benzoyl chloride as a yellow crystalline solid. MS (ES) m/z 550.2 ([M+H]⁺); HRMS: calcd for C₃₁H₃₆FN₃O₅+H+, 550.2712; found (ESI, [M+H]⁺), 550.2717.

### Example 32

### Characterization of Compounds for Gal4/hFXR and Gal4/mFXR Fusion Protein Agonist Activity in Human 293 Cells

### Materials and Methods:

Assay Medium: Phenol red free high glucose Dulbecco's modified Eagle's medium with sodium pyruvate (Cellgro, #17-205-CV) supplemented with 10% fetal bovine serum (Gibco, 16000-044), 1% glutamax (Gibco, 35050-061), 100 units/mL penicillin and 100 μg/mL streptomycin (Gibco, 15140-122).
Culturplate-96 (PerkinElmer, 6005688)
Lysis buffer (Promega, E3971)
Luciferase assay reagent (Promega E1483)

### Procedure:

### Day 1.

1. Compounds to be tested were prepared as 2× stocks in assay medium.
2. Human 293 stable clone 2 expressing Gal4/hFXR or Gal4/mFXR fusion protein were thawed from frozen stock vials, added to 9 ml of assay medium, and centrifuged at 700 rpm in a Beckman Allegra 6R centrifuge for 10 minutes. The supernatant was removed and the cells were resuspended in 1 ml assay medium. The cells were counted and diluted in assay medium to 200,000 cells per ml. The cells were then plated at 10,000 cells per well in Culturplate-96 plates in 50 uL assay medium. The cells were incubated at 37° C. for approximately 1 hour.
3. 50 uL of 2× compounds in assay medium at 37° C. were added to each well. All assays included 1 uM GW4064 (Maloney et al., J. Med. Chem., 2000, 43(16), 2971-2974) as a reference standard.
4. Cells were incubated for 24 hours at 37° C.

### Day 2.

5. The medium was removed, and the cells were lysed in 25 uL lysis buffer (Promega, E3971).
6. The plates were analyzed for luciferase activity with luciferase assay reagent (Promega E1483). Plates were read on Victor³V instrument using the protocol “Shuguang Luciferase assay” (dispense volume=100 uL, plate type=“Packard Viewplate”, measurement height=8 mm from bottom of plate, 5 second read per well).

### Analysis Of Results:

1. For agonist single point screening, data were analyzed in Excel. Each compound was tested in triplicate.
2. For agonist potency determinations, statistical analysis of the data was performed using a customized Excel/SAS program. Dose response curves were generated using a four parameter (min, max, slope, and EC₅₀ where EC₅₀ is defined as the concentration which corresponds to midway between the estimated max and min) logistic model using log-transformed data (data was transformed on both sides with known lambda=0).
3. Results for exemplary compounds of the invention are shown in Table 1 below (potency ranges are as follows: A=0.001-0.1 uM; B=0.1-0.5 uM; C=0.5-1.0 uM; and D=1.0-10 uM):

TABLE 1

| Example | Potency Range |
|---|---|
| 1 | A |
| 2 | B |
| 3 | C |
| 4 | B |
| 5 | B |
| 6 | C |
| 7 | C |
| 8 | C |
| 9 | D |
| 10 | B |
| 11 | D |
| 12 | D |
| 13 | B |
| 14 | D |
| 15 | B |
| 16 | D |
| 17 | C |
| 18 | C |
| 19 | C |
| 20 | C |
| 21 | C |
| 22 | D |
| 23 | B |
| 24 | B |
| 25 | B |
| 26 | D |
| 27 | D |
| 28 | D |
| 29 | D |
| 30 | B |
| 31 | B |

### Example 33

### Characterization of Compounds Using Fluorescence Polarization (FP) Binding Assay

Plates: Nunc 384 black shallow well plates (available from VWR, #267461)
FP buffer: PBS (available from Gibco #14190) with Ca⁺⁺ and Mg⁺⁺. Kept at 4° C. Aliquot amount needed per day and add:
CHAPS to 1 mM final(Add 33.3 μL 0.3M stock to 10 mL buffer.)
DTT to 5 mM final(Add 50 μL of 1M stock to 10 mL buffer.)
His-FXR LBD: FXR-LBD (amino acid 248-476 of FXR) with N-terminal His tag expressed from E. coli at 18 μM. Use at 20 nM final.
Fitc tagged compound: use at 0.2 nM.

Dose response experiments were performed from 10 μM to 0.001 μM with log dilutions in triplicate (264 wells) in a total reaction volume of 25 μL. 5 μl of compound plus 7.5 μl FITC labeled compound were added to achieve a 0.2 nM final solution and 12.5 μl of FXR was added to achieve a 20 nM final concentration. Plates were incubated at room temperature for 2 hours protected from light. Plates were read on PE Invision Multilabel Reader set up for fluorescence polarization (Florescein Dual setting with exitation 1 at 480 nM and exitation 2 at 535 nM).

Experimental data for exemplary compounds are shown in Table 2 below.

### Example 34

### Measurement of Progesterone Receptor (PR) Antagonism

### Materials And Methods:

### A. Reagents:

### Culture Medium:

DMEM:F12 (1:1) (GIBCO, BRL) supplemented with 5% (v/v) charcoal stripped fetal bovine serum (not heat-inactivated), 100 U/ml penicillin, 100 jig/ml streptomycin, and 2 mM GlutaMax (GIBCO, BRL).

### Alkaline Phosphatase Assay Buffer:

I. 0.1M Tris-HCl, pH 9.8, containing 0.2% Triton X-100
II. 0.1 M Tris-HCl, pH 9.8, containing 4 mM p-nitrophenyl phosphate (Sigma).

### B. Cell Culture and Treatment:

Frozen T47D cells were thawed in a 37° C. water bath and diluted to 280,000 cells/ml in culture medium. To each well in a 96-well plate (Falcon, Becton Dickinson Labware), 180 μl of diluted cell suspension was added. Twenty μl of reference or test compounds diluted in the culture medium were then added to each well. When testing for progestin antagonist activity, reference antiprogestins or test compounds were added in the presence of 1 nM progesterone. The cells were incubated at 37° C. in a 5% CO₂/humidified atmosphere for 24 hours.

### C. Alkaline Phosphatase Enzyme Assay:

At the end of treatment, the medium was removed from the plate. Fifty μl of assay buffer I was added to each well. The plates were shaken in a titer plate shaker for 15 mm. Then 150 μl of assay buffer II was added to each well. Optical density measurements were taken at 5 min intervals for 30 min at a test wavelength of 405 nM. Data was expressed as percent inhibition of the enzyme activity compared to 1 nM progesterone (antagonist mode).

Experimental data for exemplary compounds are shown in Table 2 below.

### Example 35

### Measurement of Mouse Serum Triglyceride (TG) and Cholesterol Levels

Eight-week old LDLR −/−mice were purchased from Jackson Laboratories and maintained on a chow diet. Some LDLR −/−mice were fed a western diet (AlN-76A; Purina Test Diets) as indicated. All mice were treated by daily oral gavage with vehicle or varying concentrations of compound for 7 days. On the last day after the final dose, the food was removed to allow a 3 hr fast and serum was harvested for analysis. Serum TG and cholesterol levels were determined using a Roche 912 clinical chemistry analyzer and expressed as mg/dL.

Experimental data for exemplary compounds are shown in Table 2 below.

Table 2 below includes data obtained using the assays described above as well as data obtained using standard assays well known to those of the art (including for example, hERG function, solubility, and the like) for the compound of Example 30 (isopropyl 8-fluoro-1,1-dimethyl-3-[4-(3-morpholin-4-ylpropoxy)benzoyl]-1,2,3,6-tetrahydroazepino[4,5-b]indole-5-carboxylate), Reference Compound 1 (1-methylethyl-1,1-dimethyl-3-({4-[(3-morpholin-4-ylpropyl)oxy]phenyl}carbonyl)-1,2,3,6-tetrahydroazepino[4,5-b]indole-5-carboxylate, see WO2007/070796) and Reference Compound 2 (isopropyl-3-(3,4-difluorobenzoyl)-1,1-dimethyl-1,2,3,6-tetrahydroazepino[4,5-b]indole-5-carboxylate, see WO03/099821).

TABLE 2

| | | Reference | Reference |
|---|---|---|---|
| | Example 30 | Compound 1 | Compound 2 |
| hFXR agonist EC₅₀ (eff. @ 30 μM) | 88 nM (116%) | 203 nm | 16 nM (179%) |
| | | (164%) | |
| hFXR binding IC₅₀ (μM) | 48 nM | 68 nM | 27 nM |
| mFXR agonist EC₅₀ (eff. @ μM) | 58 nM (107%) | 90 nM (87%) | 153 nM 174%) |
| hPXR-CYP3A4 EC₅₀ | 11.5 μM (46%) | | |
| PR Antagonism (Alk. Phos. Assay @ 10 μM) | 250 nM (95%) | 455 nM (90%) | 44% @ 10 μM |
| hERG function (% Inh. @ 10 μM) | >30 μM | 5.7 μM | >10 μM |
| 2% Tween/0.5% MC aq. sol. (mg/mL) | 0.20 | 0.19 | 0.01 |
| 2% Tween/0.5% MC aq. sol. (μM) | 355.0 | 332.0 | 22.0 |
| Solubility in pH 7.4 buffer (μg/mL) | 0.0 | 0.0 | 0.0 |
| PAMPA Permeability (Pe × 10⁻⁶) | 0.66 | 0 | 0 |
| Caco2 Permeability (Pe × 10⁻⁶) A-B, B-A | Low (0.8, 0.8) | ND | Low (0.5, 0.5) |
| Transport mechanism | passive | passive | passive |
| MLM (t_{1/2}) | 29 min | 8 min | 13 min |
| HLM (t_{1/2}) | >30 min | 16 min | 18 min |
| CYP450 3A4 % I @ 3 μM | 32% | 48% | 43% |
| CYP450 2D6 % I @ 3 μM | 4% | 6% | 5% |
| CYP450 2C9 % I @ 3 μM | 64% | 38% | 24% |
| Bioavailability (dosed in aqueous MC/Tween) | 21% | 15% | poor |
| Half life | 6.4 h | 4.1 h | not calculated |
| Reduction of Triglycerides in a LDLR KO | 78% | 59% | 71% |
| Mouse Model (Male) @ 10 mpk | | | |
| Reduction of Triglycerides in a LDLR KO | 65% | 25% | 39% |
| Mouse Model (Female) @ 10 mpk | | | |
| Reduction of Total Cholesterol in a LDLR KO | 74% | 49% | 47% |
| Mouse Model (Male) @ 10 mpk | | | |
| Reduction of Total Cholesterol in a LDLR KO | 60% | 40% | 36% |
| Mouse Model (Female) @ 10 mpk | | | |

As those skilled in the art will appreciate, numerous changes and modifications may be made to the embodiments of the invention without departing from the spirit of the invention. It is intended that all such variations fall within the scope of the invention. It is intended that each of the patents, applications, and printed publications including books mentioned in this patent document be hereby incorporated by reference in their entirety.

1. A compound of Formula I 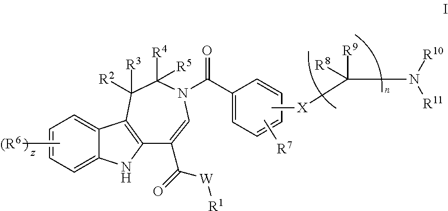
or a pharmaceutically acceptable salt thereof, wherein
W is chosen from O and NH;
X is chosen from O and CR⁸R⁹;
n is 2, 3 or 4 when X is equal to O, or
n is 0, 1, 2, 3 or 4 when X is equal to CR⁸R⁹;
z is 1 or 2;
R¹ is chosen from optionally substituted C₁-C₂₀ alkyl, optionally substituted C₃-C₁₀ cycloalkyl, optionally substituted 3- to 12-membered heterocyclyl, optionally substituted 6- to 14-membered aryl, and optionally substituted 5- to 15-membered heteroaryl;
R², R³, R⁴, and R⁵ are independently chosen from hydrogen and optionally substituted C₁-C₂₀ alkyl, or any two of R², R³, R⁴ and R⁵, together with the atoms to which they are attached, form an optionally substituted C₃-C₁₀ cycloalkyl or optionally substituted 3- to 12-membered heterocyclyl ring;
R⁶, at each occurrence, independently is chosen from halogen, optionally substituted C₁-C₂₀ alkyl, hydroxyl, optionally substituted C₁-C₆ alkoxy and cyano;
R⁷ is chosen from hydrogen, halogen, optionally substituted C₁-C₂₀ alkyl, hydroxyl, optionally substituted C₁-C₆ alkoxy and cyano;
R⁸ and R⁹, at each occurrence, are independently chosen from hydrogen, fluoro, and C₁-C₂₀ alkyl; and
R¹⁰ and R¹¹ are independently chosen from hydrogen, optionally substituted C₁-C₂₀ alkyl, optionally substituted C₃-C₁₀ cycloalkyl and optionally substituted 3- to 12-membered heterocyclyl, or R¹⁰ and R¹¹ together with the atoms to which they are attached, form an optionally substituted 3- to 12-membered heterocyclyl ring containing 1 or 2 heteroatoms including the nitrogen through which they are attached.

2. The compound of claim 1, or a pharmaceutically acceptable salt thereof, wherein W is O.

3. The compound of claim 1, or a pharmaceutically acceptable salt thereof, wherein R⁴ and R⁵ are each hydrogen.

4. The compound of claim 1, or a pharmaceutically acceptable salt thereof, wherein the benzoyl group is meta- or para-substituted with
—X—(CR⁸R⁹)ₙ—NR¹⁰R¹¹.

5. The compound of claim 1, or a pharmaceutically acceptable salt thereof, having Formula IV:

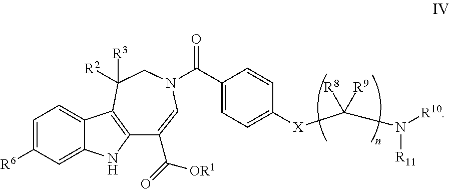

6. The compound of claim 1, or a pharmaceutically acceptable salt thereof, wherein R¹ is optionally substituted C₁-C₂₀ alkyl.

7. The compound of claim 6, or a pharmaceutically acceptable salt thereof, wherein R¹ is iso-propyl.

8. The compound of claim 1, or a pharmaceutically acceptable salt thereof, wherein R² and R³ are independently chosen from hydrogen and optionally substituted C₁-C₂₀ alkyl.

9. The compound of claim 8, or a pharmaceutically acceptable salt thereof, wherein R² and R³ are each methyl.

10. The compound of claim 1, or a pharmaceutically acceptable salt thereof, wherein R⁶ is chosen from halogen and cyano.

11. The compound of claim 10, or a pharmaceutically acceptable salt thereof, wherein R⁶ is fluoro.

12. The compound of claim 1, or a pharmaceutically acceptable salt thereof, wherein R¹⁰ and R¹¹ together with the atoms to which they are attached, form an optionally substituted 5- to 7-membered heterocyclyl ring containing 1 or 2 heteroatoms including the nitrogen through which they are attached.

13. The compound of claim 12, or a pharmaceutically acceptable salt thereof, wherein R¹⁰ and R¹¹ together with the atoms to which they are attached, form an optionally substituted 5- to 7-membered heterocyclyl ring chosen from morpholinyl, piperidinyl, piperazinyl, 4-piperidonyl, pyrrolidinyl, pyrazolidinyl, quinuclidinyl, thiazolidinyl, tetrahydrofuryl, tetrahydropyranyl, thiamorpholinyl, thiamorpholinyl sulfoxide, and thiamorpholinyl sulfone.

14. The compound of claim 13, or a pharmaceutically acceptable salt thereof, wherein R¹⁰ and R¹¹ together with the atoms to which they are attached, form a morpholinyl ring.

15. A compound selected from:
isopropyl 8-fluoro-1,1-dimethyl-3-[4-(morpholin-4-ylmethyl)benzoyl]-1,2,3,6-tetrahydroazepino[4,5-b]indole-5-carboxylate;
isopropyl 8-fluoro-1,1-dimethyl-3-[4-(2-morpholin-4-ylethyl)benzoyl]-1,2,3,6-tetrahydroazepino[4,5-b]indole-5-carboxylate;
isopropyl 8-fluoro-1,1-dimethyl-3-[4-(3-morpholin-4-ylpropoxy)benzoyl]-1,2,3,6-tetrahydroazepino[4,5-b]indole-5-carboxylate;
isopropyl 8-fluoro-1,1-dimethyl-3-[4-(2-morpholin-4-ylethoxy)benzoyl]-1,2,3,6-tetrahydroazepino[4,5-b]indole-5-carboxylate;
isopropyl 1,1-dimethyl-3-[4-(3-pyrrolidin-1-ylpropoxy)benzoyl]-1,2,3,6-tetrahydroazepino[4,5-b]indole-5-carboxylate;
isopropyl 1,1-dimethyl-3-{4-[2-(4-methylpiperazin-1-yl)ethoxy]benzoyl}-1,2,3,6-tetrahydroazepino[4,5-b]indole-5-carboxylate;
isopropyl 1,1-dimethyl-3-{4-[3-(4-methylpiperazin-1-yl)propoxy]benzoyl}-1,2,3,6-tetrahydroazepino[4,5-b]indole-5-carboxylate;
isopropyl 1,1-dimethyl-3-[3-(2-pyrrolidin-1-ylethoxy)benzoyl]-1,2,3,6-tetrahydroazepino[4,5-b]indole-5-carboxylate;
isopropyl 1,1-dimethyl-3-{3-[2-(4-methylpiperazin-1-yl)ethoxy]benzoyl}-1,2,3,6-tetrahydroazepino[4,5-b]indole-5-carboxylate;
isopropyl 1,1-dimethyl-3-[3-(3-pyrrolidin-1-ylpropoxy)benzoyl]-1,2,3,6-tetrahydroazepino[4,5-b]indole-5-carboxylate;
isopropyl 1,1-dimethyl-3-{3-[3-(4-methylpiperazin-1-yl)propoxy]benzoyl}-1,2,3,6-tetrahydroazepino[4,5-b]indole-5-carboxylate;
isopropyl 1,1-dimethyl-3[4-(morpholin-4-ylmethyl)benzoyl]-1,2,3,6-tetrahydroazepino[4,5-b]indole-5-carboxylate;
isopropyl 1,1-dimethyl-3-[4-(2-morpholin-4-ylethyl)benzoyl]-1,2,3,6-tetrahydroazepino[4,5-b]indole-5-carboxylate;
isopropyl 3-(3-{[(2R,6R)-2,6-dimethylmorpholin-4-yl]methyl}benzoyl)-1,1-dimethyl-1,2,3,6-tetrahydroazepino[4,5-b]indole-5-carboxylate;
isopropyl 3-(3-{[(2R,6S)-2,6-dimethylmorpholin-4-yl]methyl}benzoyl)-1,1-dimethyl-1,2,3,6-tetrahydroazepino[4,5-b]indole-5-carboxylate;
isopropyl 3-(3-{[(3R,5S)-3,5-dimethylpiperazin-1-yl]methyl}benzoyl)-1,1-dimethyl-1,2,3,6-tetrahydroazepino[4,5-b]indole-5-carboxylate;
isopropyl 3-{3-[(4-carbamoylpiperidin-1-yl)methyl]benzoyl}-1,1-dimethyl-1,2,3,6-tetrahydroazepino[4,5-b]indole-5-carboxylate;
isopropyl 1,1-dimethyl-3-{3-[(4-morpholin-4-ylpiperidin-1-yl)methyl]benzoyl}-1,2,3,6-tetrahydroazepino[4,5-b]indole-5-carboxylate;
isopropyl 1,1-dimethyl-3-[3-(1,3-thiazolidin-3-ylmethyl)benzoyl]-1,2,3,6-tetrahydroazepino[4,5-b]indole-5-carboxylate;
isopropyl 3-[3-(1,4′-bipiperidin-li-ylmethyl)benzoyl]-1,1-dimethyl-1,2,3,6-tetrahydroazepino[4,5-b]indole-5-carboxylate;
isopropyl 1,1-dimethyl-3-{3-[(4-pyrrolidin-1-ylpiperidin-1-yl)methyl]benzoyl}-1,2,3,6-tetrahydroazepino[4,5-b]indole-5-carboxylate;
isopropyl 3-(3-{[(3R,5S)-3,5-dimethylpiperidin-1-yl]methyl}benzoyl)-1,1-dimethyl-1,2,3,6-tetrahydroazepino[4,5-b]indole-5-carboxylate;
isopropyl 3-(3-{[(3S,5S)-3,5-dimethylpiperidin-1-yl]methyl}benzoyl)-1,1-dimethyl-1,2,3,6-tetrahydroazepino[4,5-b]indole-5-carboxylate;
isopropyl 1,1-dimethyl-3-[3-(thiomorpholin-4-ylmethyl)benzoyl]-1,2,3,6-tetrahydroazepino[4,5-b]indole-5-carboxylate,
isopropyl 3-(3-{[(2S,5S)-2,5-dimethylpyrrolidin-1-yl]methyl}benzoyl)-1,1-dimethyl-1,2,3,6-tetrahydroazepino[4,5-b]indole-5-carboxylate,
isopropyl 3-{3-[(cyclohexylamino)methyl]benzoyl}-1,1-dimethyl-1,2,3,6-tetrahydroazepino[4,5-b]indole-5-carboxylate;
isopropyl 3-(3-{[cyclohexyl(methyl)amino]methyl}benzoyl)-1,1-dimethyl-1,2,3,6-tetrahydroazepino[4,5-b]indole-5-carboxylate;
isopropyl 3-{3-[(4-hydroxypiperidin-1-yl)methyl]benzoyl}-1,1-dimethyl-1,2,3,6-tetrahydroazepino[4,5-b]indole-5-carboxylate;
isopropyl 3-{3-[(3-carbamoylpiperidin-1-yl)methyl]benzoyl}-1,1-dimethyl-1,2,3,6-tetrahydroazepino[4,5-b]indole-5-carboxylate;
isopropyl 1,1-dimethyl-3-{3-[(piperidin-4-ylamino)methyl]benzoyl}-1,2,3,6-tetrahydroazepino[4,5-b]indole-5-carboxylate; and
isopropyl 3-{3-[(1,1-dioxidothiomorpholin-4-yl)methyl]benzoyl}-1,1-dimethyl-1,2,3,6-tetrahydroazepino[4,5-b]indole-5-carboxylate,
or a pharmaceutically acceptable salt thereof.

16. A pharmaceutical composition comprising a therapeutically effective amount of a compound of claim 1, or a pharmaceutically acceptable salt thereof, and a pharmaceutically acceptable carrier or excipient.

17. A method of treating, preventing, inhibiting, or ameliorating one or more symptoms of a disease or disorder in which nuclear receptor activity is implicated, comprising administering to a subject in need thereof an effective amount of a compound of claim 1, or a pharmaceutically acceptable salt thereof.

18. The method according to claim 17 wherein the nuclear receptor is farnesoid X receptor.

19. The method according to claim 17, wherein the disease or disorder is selected from the group consisting of hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis, obesity, cancer, cholesterol gallstone disease, and nonalcoholic fatty liver disease.

20. A method of reducing plasma cholesterol levels; reducing plasma triglyceride levels; treating, preventing, inhibiting or ameliorating one or more symptoms of a disease or disorder which is affected by abnormal cholesterol, triglyceride, or bile acid levels; modulating cholesterol metabolism, catabolism, synthesis, absorption, reabsorption, secretion or excretion in a mammal; treating at least one disease state characterized by elevated expression of the Lectin-like Oxidized Low-density Lipoprotein Receptor 1 (LOX-1); or treating at least one condition that can be treated by elevating the vitamin D receptor (VDR) activity level in a patient; said method comprising administering to a subject in need thereof an effective amount of a compound of claim 1, or a pharmaceutically acceptable salt thereof.

21. A method for modulating farnesoid X receptor activity comprising contacting a cell with a compound of claim 1, or a pharmaceutically acceptable salt thereof.
